(19) United States
(12) Patent Application Publication
(10) Pub. No.: US 2026/0137766 A1
(43) Pub. Date: May 21, 2026

(54) LIPOSOME FORMULATIONS FOR TREATMENT OF ACTIVE TUBERCULOSIS

(71) Applicant: ARCHIVEL FARMA, S.L., Badalona (ES)

(72) Inventors: Pere Joan CARDONA IGLESIAS, Barcelona (ES); Luis RUIZ-AVILA, Badalona (ES); Alexandra JIMENEZ-MELSIÓ, Badalona (ES); Maria Mercè AMAT FABREGAT, Barcelona (ES)

(21) Appl. No.: 19/120,715

(22) PCT Filed: Oct. 12, 2022
(86) PCT No.: PCT/EP2022/078375

§ 371 Date: Apr. 11, 2025

(30) Foreign Application Priority Data
Oct. 14, 2021 (EP) 21382926.0

(51) Int. Cl. A61K 39/04; A61K 9/127; A61K 31/133; A61K 31/422; A61K 31/44; A61K 31/4409; A61K 31/4709; A61K 31/496; A61K 31/4965; A61K 31/5383; A61K 31/7036; A61K 47/26

(52) U.S. Cl. CPC A61K 39/04; A61K 9/127; A61K 31/133; A61K 31/422; A61K 31/44; A61K 31/4409; A61K 31/4709; A61K 31/496; A61K 31/4965; A61K 31/5383; A61K 31/7036; A61K 47/26

(57) ABSTRACT

The present invention relates to the use of a therapeutic agent based on cell wall fragments of a virulent strain of Mycobacterium tuberculosis-complex for the preparation of a drug for the treatment of patients with active tuberculosis.

### FIELD OF THE INVENTION

The present invention relates to the use of a therapeutic agent based on cell wall fragments of a virulent strain of Mycobacterium tuberculosis-complex for the preparation of a drug for the treatment of patients with active tuberculosis. This can be standalone treatment or concomitantly with chemotherapy, for example antibiotic treatment.

### BACKGROUND OF THE INVENTION

Tuberculosis (TB) is a chronic infectious disease caused by the Mycobacterium tuberculosis complex (MTB-C) bacilli, which include, for example, the species M. tuberculosis, M. bovis, M. microti and M. africanum. According to the World Health Organization (WHO), in 2019, about 10 million people developed TB and about 1.4 million died.

One widely used vaccine based on an attenuated variant of M. bovis bacteria is known as BCG (Bacillus Calmette-Guerin). Further vaccines against TB based on cell wall fragments of virulent or avirulent strains of Mycobacterium are described in the state of the art.

A particularly effective immunotherapeutic agent based on cell wall fragments of a virulent strain of Mycobacterium tuberculosis-complex was previously developed by the inventors of the present invention (EP1090318B1).

Treatment of patients with active tuberculosis usually involves prolonged treatment with multiple drugs, for example isoniazid, rifampicin, pyrazinamide and ethambutol (HRZE). As a result, drug resistance is a growing problem and the cases of multidrug-resistant tuberculosis (MDR-TB) are rising.

Consequently, there is an urgent need for more effective treatments for those patients with active TB.

### SUMMARY OF THE INVENTION

The inventors have found that an agent based on cell wall fragments of a virulent strain of Mycobacterium tuberculosis-complex is highly effective in the treatment of patients with active TB, having bactericidal activity both alone and in conjunction with chemotherapy. As stand-alone treatment the agent has similar efficacy to chemotherapy at the beginning of active TB treatment. In conjunction with chemotherapy the agent provides a strong synergistic effect. Neither could have been predicted from the prior art, which additionally strongly suggests that such agent cannot be used in subjects with active TB due to severe side effects.

### Definitions

“FCMtb” stands for fragments from a Mycobacterium tuberculosis-complex (MTB-C) strain. This is the drug substance of the liposome formulation (drug product) of the present invention.

“particle size” refers to, if not otherwise specified, the diameter of the particles. Where the particle size cannot be determined exactly, the approximate particle size is meant.

“z-average” is the average particle size, determinable as described in the material and methods section.

### Abbreviations

BCG Bacille Calmette-Guérin vaccine
CFU Colony forming units
CMCiB Centre de Medicina Comparativa I Bioimatge
de Catalunya
DP Drug product
DS Drug substance
ELISA Enzyme-linked immunosorbent assay
ELISPOT Enzyme-linked immunospot assay
EMEA European Medicines Agency
FCMtb Fragments of M. tuberculosis cells
FIM First in man
HMtb Harvest from Mtb Expansion culture
IFN-γ Interferon gamma
IGTP Institut Germans Trias I Pujol
IMP Investigational medicinal product
IPC In process controls
LCS Liposome concentrate suspension
LS Liposomal suspension
LTBI Latent tuberculosis infection
LPS Lipopolysaccharide
Mtb Mycobacterium tuberculosis
Mtb-C Mycobacterium tuberculosis-complex
NZB New Zealand Black
NZW New Zealand White
PPD Purified protein derivative
q.s. Quantum sufficit
TB Tuberculosis
TST Tuberculosis skin test
UTE Unitat de Tuberculosi Experimental
WHO World Health Organization
w/v weight/volume
w/w weight/weight
WSL Working seed lot

### BRIEF DESCRIPTION OF FIGURES

FIG. 1: Flow-chart showing upstream process of the drug substance (FCMtb), including the materials and reagents involved in the process and suitable in-process controls.

FIG. 2: Flow-chart showing downstream process of the drug substance (FCMtb), including the materials and reagents involved in the process and suitable in-process controls.

FIG. 3: Protein characterisation. FIG. 3a: SDS-PAGE Coomassie Blue staining. Lane 1: Molecular weight. Lane 2: Reference FCMtb-81 batch. Lane 3: Reference FCMtb-81 batch. Lane 4: Study FCMtb-83-20° C. 12 mo stability batch. Lane 5: Study FCMtb-83-20° C. 12 mo stability batch. Lane 6: Study FCMtb-86 RT 1-month stability batch. Lane 7: Study FCMtb-86 RT 1-mo stability batch. Lane 8: Study FCMtb-86 40° C. 1-mo stability batch. Lane 9: Study FCMtb-86 40° C. 1-mo stability batch. Lane 10: Molecular weight. FIG. 3b: SDS-PAGE Silver staining. Lane 1: Molecular weight. Lane 2: Reference FCMtb-81 batch. Lane 3: Reference FCMtb-81 batch. Lane 4: Study FCMtb-83-20° C. 12 mo stability batch. Lane 5: Study FCMtb-83-20° C. 12 mo stability batch. Lane 6: Study FCMtb-86 RT 1-mo stability batch. Lane 7: Study FCMtb-86 RT 1-mo stability batch. Lane 8: Study FCMtb-86 40° C. 1-mo stability batch. Lane 9: Study FCMtb-86 40° C. 1-mo stability batch. Lane 10: Molecular weight. FIG. 3c: Western-Blot analysis for the HSP70, PstS1, 85 complex and HSP16.3 Mycobacterium tuberculosis antigens. Lane 1: Molecular weight. Lane 2: HSP16.3 antigen. Lane 3: 85A antigen. Lane 4: PstS1 antigen. Lane 5: HSP70 antigen. Lane 6: FCMtb-87. Lane 7: FCMtb-87 HMtb. Lane 8: FCMtb-86. Lane 9: FCMtb-86 HMtb. Lane 10: Molecular weight.

FIG. 4: Lipid analysis. FIG. 4a: Trehalose dimycolate TLC analysis. Lane 1: TDM standard. Lane 2: Reference FCMtb-81 batch. Lane 3. Study FCMtb-86 RT 6 mo stability batch. Lane 4. Study FCMtb-86 40° C. 6 mo stability batch. Lane 5. Study FCMtb-86-20° C. 6 mo stability batch. Lane 6: TDM standard. FIG. 4b: Mycolic acids TLC analysis. Lane 1: MA standard. Lane 2: Reference FCMtb-81 batch. Lane 3. Study FCMtb-86 RT 6 mo stability batch. Lane 4. Study FCMtb-86 RT 6 mo stability batch. Lane 5. Study FCMtb-86 40° C. 6 mo stability batch. Lane 6: Study FCMtb-86 40° C. 6 mo stability batch. Lane 7: Study FCMtb-86-20° C. mo stability batch. Lane 8: Study FCMtb-86-20° C. 6 mo stability batch. Lane 9: MA standard. FIG. 4c: Lipoarabinomannan (LAM) Western-Blot analysis of different FCMtb batches and their corresponding HMtbs. Lane 1: Molecular weight. Lane 2: LAM antigen 0.270 ug. Lane 3: LAM antigen 0.068 ug. Lane 4: LAM antigen 0.017 ug. Lane 5: FCMtb-83 batch. Lane 6: FCMtb-83 HMtb. Lane 7: FCMtb-86 batch. Lane 8: FCMtb-86 HMtb. Lane 9: Molecular weight. LM: Lipomannan.

FIG. 5: Freeze-fracturing preparation of liposomal concentrate (LCS) bulk (electronic microscopy).

FIG. 6: Flow-chart of the process according to the preferred mode of the drug product production.

FIG. 7: Example timings of drug product (RUTI) administration in example 9. RUTI was administered as a single dose containing 200 μg of FCMtb at different time points after starting the chemotherapy treatment and the sacrifice was done several different time points (see example 9 for details).

FIG. 8: Effect of treatment on bacillary load in lung (A) and spleen (B) versus the standard chemotherapy treatment (SCT) control group. Each point represents CFUs per animal. Mean. Statistical significances were tested using unpaired t-test, *p<0.05.

FIG. 9: Effect of treatment on lung pathology; Histometric analysis. Each point represents % Damage lung per animal. Mean. Statistical significances were tested using unpaired t-test, *p<0.05.

FIG. 10: Effect of treatment on T cell immunoresponse. Splenocytes were obtained from each spleen and incubated 24 h in presence of PPD. Each point represents IFN-γ spot number per 106 cells. Mean. Statistical significances were tested using unpaired t-test, *p<0.05.

FIG. 11: Effect of treatment on T cell immunoresponse. Splenocytes were obtained from each spleen and incubated 24 h in presence of ESAT-6. Each point represents IFN-γ spot number per 106 cells. Mean. Statistical significances was tested using unpaired t-test, *p<0.05.

FIG. 12: Effect of treatment on T cell immunoresponse. Splenocytes were obtained from each spleen and incubated 24 h in presence of HSP16. Each point represents IFN-γ spot number per E+06 cells. Mean. Statistical significances was tested using unpaired t-test.

FIG. 13: FIG. 13 a/b/c show different example schedules of administration and sacrifice used in example 10.

FIG. 14: Effect of treatment on bacillary load in lung. Each point represents CFUs per animal. Median±interquartile range for each treatment group. Statistical significances was tested using Mann-Whitney test, non-parametric *p<0.05, p<** 0.01.

FIG. 15: Effect of treatment on bacillary load in spleen. Each point represents CFUs per animal. Median±interquartile range for each treatment group. Statistical significances was tested using Mann-Whitney test, non-parametric *p<0.05, p<** 0.01.

FIG. 16: Effect of treatment on lung pathology; Histometric analysis. Each point represents percentage lung damage per animal. Median±interquartile range for each treatment group. Statistical significances was tested using Mann-Whitney test, non-parametric *p<0.05, p<** 0.01.

FIG. 17: Effect of treatment on T cell immunoresponse. Splenocytes were obtained from each spleen and incubated 24 h in presence of PPD or ESAT-6. Each point represents IFN-γ spot number per E+06 cells. Median±interquartile range for each treatment group. Statistical significances was tested using Mann-Whitney test, non-parametric *p<0.05, p<** 0.01.

FIG. 18: Effect of treatment on T cell immunoresponse. Splenocytes were obtained from each spleen and incubated 24 h in presence of HSP16 or PSTS1. Each point represents IFNγ spot number per E+06 cells. Mean. Statistical significances was tested using unpaired t-test, *p>0.05.

### DETAILED DESCRIPTION

The inventors have surprisingly found that administration of an immunotherapeutic agent based on cell wall fragments of a virulent strain of Mycobacterium tuberculosis-complex previously exclusively used for the prophylaxis of TB, i.e. administered before the patient developed active TB, or after the administration of chemoprophylaxis is an effective treatment for those patients with active TB which have yet to receive chemotherapy.

This agent shows bactericidal effect both as standalone treatment and synergistically by improving the efficacy of standard TB chemotherapy when administered before or concomitantly.

When administered to a laboratory animal (mice) who has already developed active TB which has not yet received chemotherapy, the agent as stand-alone treatment immediately reduces bacterial load by a factor of 15 during the first week, and has similar efficiency as standard chemotherapy. Further, when administered to a subject infected with tuberculosis before or concomitantly with chemotherapy, there is a clear synergistic effect. In this case, the bacterial load is reduced by a factor of 225 during the first week, an order of magnitude stronger than expected from a simple additive effect.

This novel use of the agent in the treatment of TB will, for example, allow for the reduced use of chemotherapy and improved treatment for patients.

This solution could not have been expected from the available prior art primarily because it was common general knowledge that administration of such agents to subjects with active TB will result in strong adverse side effects. This phenomenon is particularly well-studied in the context of the use of tuberculins in the absence of prior chemotherapy. In this context, the “Koch phenomenon” describes a skin reaction that appears in guinea pigs suffering from TB within a few days at the site where living tubercle bacilli or extracts of tubercle bacilli (tuberculins) are inoculated.

Without wishing to be bound to theory, the Koch phenomenon describes an adverse reaction of subjects already ill with TB which are inoculated with an agent based, for example, on cell wall fragments of a virulent strain of Mycobacterium tuberculosis-complex.

In such cases, the region of inoculation becomes hard and darkened. This effect is not restricted to the inoculation point but spreads out to a diameter of 0.5-1 cm. The altered skin can also become necrotic and slough off, leaving a flat ulcer which usually heals rapidly and permanently without involvement of the neighbouring lymph nodes. This effect is not caused exclusively by living tubercle bacilli but also by killed ones or fragments thereof.

Consequently, use of agents as described herein in patients with active TB in the absence of prior treatment with chemotherapy was highly discouraged from the prior art, as presumably would not only affect the site of the inoculation but the injured lungs.

Additionally, the agent presented in EP1090318B1 or EP2090318B1 was developed and tested as a prophylactic agent for those not yet infected with TB or administered to those with latent TB after standard chemoprophylaxis.

As such, the strong effect of the agent by itself on the bacterial load of infected subjects with active TB could not have been predicted, much less the strong synergistic effect with existing chemotherapy when administered before or concomitantly. Especially the latter is a critical element of success in chemotherapy for tuberculosis, in terms of efficacy, cost-effectiveness, reduction of potential side effects by the treatment and, above all, prevention of development of multidrug-resistance.

The present invention provides a therapeutic vaccine for use in a method of treating active tuberculosis. For example, this can be an agent containing fragments from a Mycobacterium tuberculosis-complex for use in a method of treating active tuberculosis, not limited to those particular embodiments disclosed herein.

More specifically, the invention provides liposome formulations (drug product) comprising fragments from a Mycobacterium tuberculosis-complex (MTB-C) strain (FCMtb, drug substance) and a liposome forming agent for use in a method of treating active tuberculosis.

In several examples of the present invention the drug product is referred to as “RUTI”, a particularly preferred embodiment of the liposome formulation of the present invention.

Unless expressly specified otherwise, the term “comprising” is used in the context of the present application to indicate that further members may optionally be present in addition to the members of the list introduced by “comprising”.

It is, however, contemplated as a specific embodiment of the present invention that the term “comprising” encompasses the possibility of no further members being present, i.e. for the purpose of this embodiment “comprising” is to be understood as having the meaning of “consisting of”.

This detailed description discloses specific and/or preferred variants of the individual features of the invention. The present invention also contemplates as particularly preferred embodiments those embodiments, which are generated by combining two or more of the specific and/or preferred variants described for two or more of the features of the present invention.

It is generally accepted that a liposomation process generates a lipidic environment, facilitating the solubility and leading to a suspension of substances, such as FCMtb (the drug substance of the present invention). Liposomes within the meaning of this invention may be unilamellar, multilammellar or combinations thereof.

FCMtb (drug substance) can be of any type of substance derived from the MTB-C strain, whereby fragments derived from proteins and/or lipids are preferred. FCMtb within the sense of this application is typically a mixture of different protein antigens and lipids from MTB-C cells. The cell fragments may be obtained by any method known to the person skilled in the art suitable for fragmenting microbial or bacterial cells, such as specifically MTB-C cells, for example homogenisation. The homogenisation can be carried out by means of ultrasound sonication, or by means of the use of small beads of approximately 0.1 mm in diameter, for example, silica or zirconia/silica beads, together with a mechanical homogenizer.

A mechanical homogenizer that can be used, for example, the BioSpec BeadBeater® model. The MTB-C cells are broken by means of this homogenisation process, so that small cell fragments, typically including nano cell wall fragments, are obtained. A typically relevant feature of the manufacturing of the cell fragments is the “detoxification” of the cell wall fragments by delipidation, well known to the person skilled in the art, a process that allows removing the endotoxin-like molecules. The FCMtb is therefore preferably detoxified, pasteurized and lyophilized. The final drug product, i.e. the liposome formulation of the present invention, most preferably RUTI as described in the examples, is prepared as a liposomal formulation of FCMtb in sucrose, filtered through 0.22 nm. The final drug product, most preferably RUTI, can optionally be lyophilised to facilitate the storage thereof. To that end, the final drug product, most preferably RUTI, can be distributed into vials and lyophilised, for example at a temperature in the range of −45° C. to 25° C. temperature and a pressure between 0.1 and 0.5 mbar, such as 0.150 mbar.

The liposomes according to this invention usually have a size distribution in which at least 99.9% (by number) are smaller than 1 μm. In a particular embodiment, the z-average size of the particles, as determinable by dynamic light scattering, is 120 nm or less, preferably 110 nm or less, more preferably 95 nm or less, and most preferably 80 nm or less. In dynamic light scattering the z-average parameter is considered a stable and important number obtainable by the technique, and the size number that is preferably used for quality control purposes. Preferably, the liposomes of the formulation according to this invention are monomodal, i.e. they show only one peak in dynamic light scattering measurements. More preferably, the liposomes of the formulation according to this invention are spherical, as can be tested by electron microscopy of freeze-fracturing preparations of the liposome formulation (drug product), as shown in Example 8. Spherical thereby means that for at least 90% of the liposome particles (by number), all surface points of the individual particle have similar or identical distance to the center of the liposome, i.e. the minimal radius of such a particle relates to the maximal radius of the same particle in a ratio of 0.6 or more, 0.7 or more, 0.8 or more or 0.9 or more. The liposome formulation (drug product) according to the present invention can comprise multilamellar or unilamellar liposomes, or a mixture thereof. In line with standard knowledge of the person skilled in the art, the dynamic light scattering measurements should be performed in a suitable buffer, i.e. a buffer which does not by itself cause disruption, disintegration or fusion of the liposomes or significantly destabilize them physically in any other way. As a rule of thumb, any buffer may be suitable as long as both ionic strength and pH value are comparable to the buffer in which the liposomes had been formed maybe suitable. Preferably, a buffer of similar or identical composition to the buffer in which the liposomes had been formed, is used.

The liposome formulation (drug product) additionally comprises 1 to 20% (w/v) sucrose, preferably 2 to 12% (w/v) sucrose, more preferably 3 to 8% (w/v) sucrose, and most preferably 4 to 6% (w/v) sucrose. Approximately 5% sucrose are particularly preferred.

It is important to note that, while each of these embodiments relating, the one relating to a particular particle size and the other relating to the presence of sucrose, may be fulfilled individually, these embodiments are not to be seen as mutually exclusive and may well occur in combination.

In one particular embodiment, the above-described liposome formulation has a z-average particle size in the range from 40 to 120 nm, preferably from 50 to 100 nm, and more preferably from 55 to 95 nm, and more preferably from 55 to 80 nm. The z-average particle size is thereby preferably measured by dynamic light scattering, as described in general above and in detail in the section “Materials and methods”.

In an alternative particular embodiment, the z-average particle size of the above-described liposome formulation may be smaller, so that the liposome formulation is an emulsion, i.e. in this particular embodiment the z-average size of the particles is preferably below 40 nm.

In a preferred embodiment of any one or more of the above-described embodiments, the liposomes of the formulation according to this invention are furthermore monodisperse, which means that no significant width of the size distribution is observed. This is technically tested by a low polydispersity index (PDI) as determined by dynamic light scattering, such as 0.400 or less, preferably 0.300 or less. Hence, the liposome formulation is a liposome formulation, wherein the polydispersity Index of the particles as determinable by dynamic light scattering is 0.400 or less, preferably 0.300 or less, and most preferably 0.250 or less.

The fragments from the Mycobacterium tuberculosis-complex (MTB-C) strain are obtainable by a process comprising an upstream process and a downstream process. For illustrative purposes, the main steps are briefly described here and particular modes of carrying out the process are given in Examples 2 and 3 below.

### Upstream Process (Example 2):

Step 1: Culture of Mtb-WSL
Step 2: Harvest of Mtb-WSL and inoculation and growth of Mtb Expansion culture
Step 3: Harvest of Mtb-Expansion culture (HMtb) and freezing

### Downstream Process (Example 3):

Step 4: Cell fragmentation and delipidation (purification)
Step 5: Pasteurization
Step 6: Filtration and filling
Step 7: Freeze-drying, encapsulation and labelling

In a more preferred embodiment of any of the above-described embodiments, the Mycobacterium tuberculosis-complex (MTB-C) strain is a virulent Mycobacterium tuberculosis-complex (MTB-C) strain. Virulent refers to the pathogenicity by case and/or the ability of the bacilli to invade the tissues of the host. The virulent strain can be any virulent strain of any of the species belonging to MTB-C, but a strain belonging to M. tuberculosis is preferred. The MTB-C strain according to this invention may be cultivated by inoculation in culture media well-known by the person skilled in the art, for example Middlebrook 7H10 or 7H11 agar, Sauton's medium or Proskauer-Beck medium. The culture of the virulent strain is preferably performed over an extended time period, such as, for example, a period equal to or greater than three weeks, preferably comprised between 3 and 4 weeks. The temperature of the culture is preferably maintained between 34° C. and 38° C. Once the culture ends, the cells are harvested and isolated using techniques well known in the art, such as those described in patent application ES2231037-A1.

The liposome agent of the liposome formulation (drug product) is preferably a hydrogenated, partially hydrogenated or non-hydrogenated phospholipid. The phospholipid used can be or comprise, for example: phosphatidylcholine, phosphatidylserine and phosphatidylinositol. Most typical is phosphatidylcholine, which can be synthesized or isolated from a variety of natural sources. Preferably the liposome forming agent is or comprises lecithin, selected from the group consisting of egg lecithin and soy lecithin. Soy lecithin is a complex mixture of phospholipids including inter alia phosphatidylcholine, and is particularly preferred. Typical lipids which may also be comprised in the formulation, either as liposome forming agent itself, or as further component, are: dicetyl phosphate (DCP), dimyristoyl phosphatidylcholine (DMPC), dimyristoyl phosphatidylglycerol (DMPG), dioleoyl phosphatidylcholine (DOPc), dioleoyl phosphatidyleth-anolamine (DOPE), dioleoyl phosphatidylserine (DOPS), dipatmitoyl phosphatidylcholine (DPPC), dipalmitoyl phosphatidylglycerol (DPPG), phosphatidylcholine (PC) and/or phosphatidylserine (PS), whereby the respective lipid may behydrogenated, partially hydrogenated or non-hydrogenated. The liposomes can be formed using conventional auxiliary lipids and techniques well-known by the person skilled in the art, such as those described in the patent application ES2231037-A1.

It is further preferred that in any of the embodiments described above, the ratio of (a): the fragments from a Mycobacterium tuberculosis-complex (MTB-C) strain and (b) the liposome forming agent, is between 0.01:1 and 1:1, preferably between 0.06:1 and 0.1:1. In a more preferred embodiment of any of the above-described, the liposome formulation additionally comprises: (d) a tensioactive agent. Generally, all types of agents capable of changing the value of surface tension may be used as tensioactive agent in the sense of this invention, but excluded are compounds which fall under the definition of the liposome-forming agent given above. Various types of tensioactive agents are known to the person skilled in the art and may be used in the liposome formulation according to the present invention. As is known to the skilled person, tensioactive agents are generally chemicals with a polar-nonpolar structure. Without wishing to be limited to any particular theory, tensioactive agents generally have the tendency to locate to the surface of particles, thereby creating a monomolecular layer on the interface that reduces the surface tension value. Tensioactive agents are also referred to as surfactants or active surface agents. In a preferred embodiment of the surfactant-containing liposome formulation, the tensioactive agent is selected from sterols and derivatives thereof, such as cholesterol, and/or bile salts or derivatives thereof, such as cholate. Particularly preferred embodiments are those wherein the tensioacive agent is selected from cholate, deoxycholate, cholesterol and cholesterol hemisuccinate. A good, but not limiting mode of carrying out the invention is where the liposomes of the formulation comprise both soy-derived lecithin and sodium cholate.

In an even more preferred embodiment, the liposome formulation comprising (d) the tensioactive agent, is a liposome formulation, wherein the ratio between (a) and (d) is between 0.05:1 and 3:5 (w/w). Various types of liposome forming agents may be used, as are well known to the person skilled in the art.

The liposomes can optionally contain additives improving their stability, for example: vitamin E, which is believed to act as a lipid antioxidant.

In a more preferred embodiment, the liposome formulation described above is a liposome formulation, wherein the fragments of MTB-C cells are or comprise cell wall fragments.

Any strain belonging to MTBC, and preferably any strain belonging to Mycobacterium tuberculosis, may be used. In another more preferred embodiment, the liposome formulation described above comprises fragments of the MTB-C strain NCTC 13536, which was deposited in 2010 at the NCTC in London (Example 1). Another strain which may be used, and fragments of which may therefore be comprised in the liposome formulation, is called H37Rv, which, for example, can be obtained from the National Collection of Type Cultures (NCTC), London, Great Britain (deposit number NC007416) and is often used by researchers in this field. It is also possible that more than one strain be used, i.e. that the liposome formulation comprises fragments of various, such as two, three, or more than three strains.

Considering the approximately 4000 putative antigens of M. tuberculosis, it is impossible to analyse the drug substance for all of these proteins. Nevertheless, certain MTB-C proteins have been shown to be relevant for the desired immune response. These are five protein bands have approximate sizes of 6, 10, 16, 30, 38, and 70 kDa (Renshaw, et al., 2005, EMBO Journal 24, 2491-2498; Singh, et al., 2005, Clin. Diagn. Lab. Immunol. 12 (2), 354-358; Rodriguez-Hernandez, et al. 2020, Biomed and Biotechnol 21 (11): 856-870; Meier et al., 2018, Frontiers in Immunology Vol9 Article 2476). Therefore, in an even more preferred embodiment, the liposome formulation described above comprises at least two, preferably three, more preferably four, more preferably five, and most preferably all of the following:
(i) a first polypeptide having a molecular weight of about 70 kDa as measured following electrophoresis on a sodium dodecylsulfate (SDS) polyacrylamide gel, wherein the first polypeptide has a mass fingerprint similar to a mass fingerprint of M. tuberculosis HSP70 protein (Rv0350),
(ii) a second polypeptide having a molecular weight of about 38 kDa as measured following electrophoresis on a sodium dodecylsulfate (SDS) polyacrylamide gel, wherein the second polypeptide has a mass fingerprint similar to a mass fingerprint of M. tuberculosis PsTS1 protein (Rv0934),
(iii) a third polypeptides having a molecular weight of about 30-34 kDa as measured following electrophoresis on a sodium dodecylsulfate (SDS) polyacrylamide gel, wherein the third polypeptides have a mass fingerprint similar to a mass fingerprint of M. tuberculosis Ag85 complex including Ag85A and Ag85B proteins (Rv 3804c and Rv 1866c, respectively),
(iv) a fourth polypeptide having a molecular weight of about 16 kDa as measured following electrophoresis on a sodium dodecylsulfate (SDS) polyacrylamide gel, wherein the fourth polypeptide has a mass fingerprint similar to a mass fingerprint of M. tuberculosis HSP16 protein (Rv 2031c),
(iv) a fifth polypeptide having a molecular weight of about 10 kDa as measured following electrophoresis on a sodium dodecylsulfate (SDS) polyacrylamide gel, wherein the fifth polypeptide has a mass fingerprint similar to a mass fingerprint of M. tuberculosis CFP10 protein (Rv3874), and
(v) a sixth polypeptide having a molecular weight of about 6 kDa as measured following electrophoresis on a sodium dodecylsulfate (SDS) polyacrylamide gel, wherein the sixth polypeptide has a mass fingerprint similar to a mass fingerprint of M. tuberculosis ESAT-6 protein (Rv3875).

In a yet more preferred embodiment thereof, the liposome formulation further comprises a lipopolypeptide having a molecular weight of about 19 kDa as measured following electrophoresis on a sodium dodecylsulfate (SDS) polyacrylamide gel, wherein the lipopolypeptide has a mass fingerprint similar to a mass fingerprint of M. tuberculosis 19 kDa lipoprotein antigen precursor LpqH (Rv 3763). The respective band can be visualized by methods known in the art, such as silver staining. The researchers of the present invention surprisingly found that this polypeptide induces a high total IgG humoral response, which may be the highest humoral response among all antigens in the formulation.

Examples of how the polypeptides or lipopolypeptides may be identified are given in Example 4.

Even more preferably, the liposome formulation described above is further characterized in that at least one of the following antigens of Mycobacterium tuberculosis, or fragment thereof, is present: HSP70, PsTS1, 85 complex, HSP16 and most preferably at least one of HSP70, PsTS1, 85 complex, HSP16. Fragment in this sense is any part, such as for example a degradation product, of any of these polypeptides. Various ways of obtaining such fragments are possible, for example chemical or enzymatic hydrolysis, whereby it is not relevant if the fragmentation had occurred purposely or not, prior to liposome formation or thereafter. It is preferred that the respective fragment can be assigned to its respective origin, such as, for example, by substantial overlap in amino acid sequence, such as at least 5, at least 10, at least 20 consecutive amino acids.

The drug substance (FCMtb) is manufactured from bacilli grown under the stressful conditions of starvation, low pO₂ and low pH, conditions achieved gradually by culturing on solid media and leading to stationary growth in which a slow metabolism makes bacilli more resistant to stress. The multiantigenic nature of DS (multiantigenic protein mixture plus lipids instead of purified antigens alone) appears to be an advantage and, in this sense, fragmentation process is selected to allow the optimal presentation of this cellular antigens' mixture.

Another relevant feature of the manufacturing of DS is the delipidation of the cell wall fragments that allows removing the endotoxin like molecules.

Under such conditions, cultured bacilli include mainly characteristics of non-replicating bacilli. The antigens present in FCMtb are thus expected to trigger a wide poli-antigenic response against active bacilli and non-replicating bacilli.

Further details regarding the production process can be found in the examples.

Mycobacterial glycolipids have long been recognized to have immunomodulatory activity, notably the induction of granulomatous responses and to exert potent adjuvant-like effects. Therefore, in a more preferred embodiment, the liposome formulation described above contains lipids which are typically found in Mycobacterium tuberculosis, or derivatives thereof, such as conjugation products like sugar conjugated lipids. Several immunogenic lipid components have been identified in M. tuberculosis samples (Brennan, Tuberculosis (Edinburgh), 2003, 83(1-3), 91-97; Chouldhary et al., 2018, Journal Immunology, 200:3053-3066) and analytical methods for their determination have been developed (electrophoresis, SDS-PAGE, thin layer chromatography, western blot). Although the isolation of each lipid component would require such an aggressive treatment that quantitative data or percentages of each component detectable in the MTB-C extract or in the liposome formulation are difficult to obtain, the qualitative characterisation shall serve to characterize a further preferred embodiment of this invention. According to this further preferred embodiment, one or more of mycolic acids, preferably belonging to any one or more of types I, III or IV is comprised. Alternatively or in addition, a sugar-conjugated mycolate, preferably trehalose dimycolate may be comprised in the formulation. Alternatively or in addition, a glycolipid lipoarabinomannan (LAM) may be comprised in the formulation. Furthermore, the multiantigenic nature of the fragments (multiantigenic protein mixture plus lipids, instead of purified antigens alone) is believed to be an advantage and therefore the cell fragmentation process can be adapted by the skilled person so as to allow the optimal cellular antigen mixture.

Homogenisation of the MTB-C cells is carried out in the presence of one or more surfactants, preferably a nonionic surfactant. Hence, the liposome formulation (drug product, preferably RUTI) described above may additionally comprise one or more such surfactants. A large number of such surfactants are within the standard knowledge of a person skilled in the art. Preferably, the nonionic surfactant used is selected from the group consisting of alkylphenol ethoxylates, and sorbitan ester ethoxylates. More preferably, the nonionic surfactant is selected from the group of octylphenol ethoxylates. Even more preferably, octylphenol ethoxylates with an ethylene oxide content comprised between 7 and 8 moles are used, which surfactants can be found on the market under the name Triton X-100. The homogenised mass containing the cell wall fragments is subjected to a conventional treatment to separate and reject the non-fragmented cells and the solubilized components. Centrifugation at different speeds and washing with buffer solution as described in patent application ES2231037-A1 can be used for example. Sediment containing the cell wall fragments is obtained after performing the mentioned purification processes. Said sediment is dispersed in phosphate-buffered saline (PBS) buffer and is subjected to a conventional treatment to ensure the complete inactivation of the MTB-C cells which may have remained viable after the fragmentation and purification process. The mentioned treatment can be a chemical process, for example by means of treatment with formaldehyde, or a physical process, for example by means of autoclaving or pasteurisation treatment. Examples of lipid characterization are given in Example 5.

In a further preferred embodiment, the above described liposome formulation additionally comprises one or more salts or solution(s) thereof, whereby the preferred salt is sodium chloride.

In an even more preferred embodiment of any of the above, the liposome formulation is freeze-dried. The liposomes can be subjected to lyophilisation to thus obtain the immunotherapeutic agent in the form of lyophilised liposomes. To that end, the dispersion can be distributed into vials and lyophilised at a low temperature, for example at a temperature in the range of −45° C. to 25° C. temperature and a pressure between 0.1 and 0.5 mbar, such as 0.150 mbar. The vials obtained after lyophilisation contain the liposome formulation suitable as immunotherapeutic agent and they are preferably stored at very low temperatures, for example at 5° C.

The invention also provides a suspension, wherein the liposome formulation (drug product, RUTI) of any of the preceding claims is reconstituted in a solvent. In a preferred embodiment, the solvent of this suspension is aqueous, more preferably and most preferably is or comprises physiological serum. Methods of suspending liposome formulations in a solvent are well known to the person skilled in the art. It is a particularly advantageous property of the formulation according to this invention that it can be suspended faster than conventional liposome formulations comprising MTB-C fragments.

One object of the invention is the provision of an agent comprising cell wall fragments of a strain of MTB-C for the preparation of a pharmaceutical composition, whereby the agent is or comprises the liposome formulation described above in any of the embodiments described or combinations thereof. The main scope of the pharmaceutical formulation/galenic formulation of the drug substance is to obtain a suspension effective and stable enough to be well recognized by the cells and which has the potential to trigger a relevant cellular immune response in a human or animal body. To that end, the invention also provides a pharmaceutical composition comprising the liposome formulation, or the suspension as described in any one or more of the embodiments described above, and a pharmaceutically acceptable carrier, excipient or diluent. Various such carriers, excipients and diluents are known to the person skilled in the art, and they are in no way limited by this disclosure. Rather, any substance suitable as carrier, excipient or diluent may be used. In a preferred embodiment, this pharmaceutical composition additionally comprises a pharmaceutically acceptable adjuvant. Adjuvant is thereby to be understood as a substance comprised in this embodiment of the invention, whereby the adjuvant is a substance capable of stimulating the immune system when applied to a human or animal body in response to the target antigen, whereby the adjuvant does not itself confer immunity. Without wishing to be limited to any particular adjuvant substance, preferred embodiments are wherein the adjuvant is an aluminium salt, such as aluminium chloride, or a mineral oil or a composition comprising mineral oil, such as incomplete Freund's adjuvant (IFA) or complete Freund's adjuvant (CFA), or an ammonium halogenide, such as an alkylated ammonium bromide, such as dimethyldioctadecylammonium bromide.

The invention also provides a product for use in a method of treatment of the human or animal body by therapy. That is, it provides the liposome formulation according to any one or more of the embodiments described above, the suspension according to any one or more of the embodiments described above, or the pharmaceutical composition according to any one or more of the embodiments described above for use in a method of treatment of the human of animal body by therapy.

The drug can be administered in a mucosa, for example, ocular, intranasal, oral, gastric, intestinal, vaginal, or urinary tract mucosa, or parenterally, for example, subcutaneously, intradermally, intramuscularly, intravenously, or intraperitoneally. Parenteral administration is preferred. In a particular embodiment, the invention provides this liposome formulation, suspension or pharmaceutical composition for injection.

In another particular embodiment, the invention provides this liposome formulation, suspension or pharmaceutical composition for use in a method of treating active tuberculosis. The inventors of the present study have found that the formulation according to the present invention is highly effective in the treatment of patients with active TB, both as stand-alone treatment and in conjunction with known chemotherapy. As stand-alone treatment the agent has similar efficacy to chemotherapy. In conjunction with chemotherapy the agent provides a strong synergistic effect.

“Chemotherapy” in the context of treatment of active TB refers to any treatment according to the WHO recommendations. The skilled person is aware which treatments are currently recommended by the WHO. In particular, this includes but is not limited to a variety of antibiotic therapies. For example, chemotherapy can include any of the following: Isoniazid, rifampin, rifapentine, rifabutine, Levofloxacin, moxifloxacin, gatifloxacin, ofloxacin, ciprofloxacin, sparfloxacin, Bedaquiline, Pretomanid, Linezolid, Clofazimine, Cycloserine, terizidone, Ethambutol, Delamanid, Pyrazinamide, imipenem, cilastatin, meropenem, ertapenem, Amikacin, Streptomycin, Ethionamide, prothionamide, P-aminosalicylic acid, Kanamycin, Capreomycin, Amoxicillin/clavulanate, ampicillin/clavulanate, gentamicine, tobramycin, clarithromycin, azithromycin, thiacetazone.

In the context of the present invention, antibiotic therapy is preferred. In a preferred embodiment the antibiotic treatment comprises at least one of ethambutol, isoniazid, pyrazinamide, rifampicin, streptomycin, amikacin, kanamycin, capreomycin, viomycin, enviomycin, ciprofloxacin, levofloxacin, moxifloxacin, ethionamide, prothionamide or cycloserineterizidone. In a particularly preferred embodiment the antibiotic treatment comprises RIMSTAR/HRZE.

“active TB” is pulmonary active TB and/or extra pulmonary active TB. Preferably, active TB is pulmonary active TB. In particular, active TB includes but is not limited to drug susceptible tuberculosis, Rifampicin resistant tuberculosis, multidrug-resistant tuberculosis or extensive drug resistant tuberculosis.

Without wishing to be bound to theory, the most relevant difference between latent tuberculosis and active tuberculosis is the expected bacillary load, being approx. 10⁵ CFU in latent at the most. Any higher bacillary load can be considered active tuberculosis. Bacillary load can be up to ×10.000 or ×100.000 times higher, i.e. up to 10⁹/10¹⁰ CFU. The most bacilli can be found in the exponential phase.

Furthermore, the skilled person is aware of further experimental methods to determine whether a patient has active TB, such as a positive smear test (due to the high bacilli number in the lung) and/or TB lesions in the lung, for example, visible on X-ray.

The suitable dose of the liposome formulation, suspension or pharmaceutical composition according to what is described above in relation to the use thereof in a method of treatment of the human body by therapy depends on several parameters, including the method of administration and the subject to be treated. In a preferred embodiment, it is for administration to the human body. In a preferred embodiment thereof, this occurs in a dose comprising 1 to 1000, preferably 3 to 250 μg/dose of FCMtb. 25 μg of FCMtb per dose is particularly preferred in humans.

In two more particular embodiments the liposome formulation, suspension or pharmaceutical composition according to what is described above in relation to the use thereof in a method of treatment of active tuberculosis is (a) for use in the absence of chemotherapy or (b) in the presence of chemotherapy. In particular, the liposome formulation may be used adjunctive to chemotherapy, by increasing the efficacy of the chemotherapy, which could reduce the length of the treatment.

The liposome formulation, suspension or pharmaceutical composition for the use thereof in a method of treating active tuberculosis can be administered in the form of a single dose or of several, such as two, three, four, five or more than five, doses, by means of the repetition at certain time intervals, both in the presence or absence of chemotherapy. Most preferably, the liposome formulation, suspension or pharmaceutical composition for the use thereof in a method of treating active tuberculosis can be administered once or twice.

In a particularly preferred embodiment the liposome formulation, suspension or pharmaceutical composition is given to humans as a single dose of 25 μg of FCMtb once during chemotherapy. This can be at any time during chemotherapy of active TB.

Generally, the intensive chemotherapy phase of treatment of active TB can vary between different types of active TB, such as drug susceptible tuberculosis, Rifampicin resistant tuberculosis, multidrug-resistant tuberculosis or extensive drug resistant tuberculosis and the liposome formulation can be administered at any point during this chemotherapy.

In another particularly preferred embodiment the liposome formulation is given as standalone treatment.

Examples 9 and 10 are examples of how the liposome formulation according to this invention may be used for the treatment of active tuberculosis in the presence or absence of chemotherapy and in different dosage schedules.

The examples together clearly show that there is a reduction in the bacillary load when the liposome formulation (drug product, RUTI) is administered alone. This effect is comparable to chemotherapy during the first week. Without wishing to be bound to theory, the fast effect suggests a mechanism of action involving both an adaptive and an innate immune response. In addition, an even more pronounced reduction in bacillary load can be observed in combination with chemotherapy. The reduction is clearly synergistic in nature.

At the same time, no pathological observation (see parameter % of lung damage area) which could be attributed to the Koch phenomenon was observed in any of the experiments. By contrast, both the formulation alone and in combination with chemotherapy (RIMSTAR) showed an improvement in % lung damage versus the control groups.

Example 11 also shows that no significant safety concerns were observed when RUTI was administered to patients with MDR-TB or DS-TB concomitant with standard antibiotic treatment (1 week or 1 month after start of antibiotic therapy).

A preferred embodiment of the use of the liposome formulation, suspension or pharmaceutical composition in the treatment of active TB is use in combination or adjunctive therapy. Medical practitioners use adjunctive therapy frequently to get better cure rates or a faster response to primary treatment. A combination therapy or adjunctive therapy involves using more than one medicine to treat the human or animal body by therapy.

A preferred embodiment of a combination therapy is one wherein the combination therapy comprises an antibiotic, preferably one or more of isoniazid and an ansamycine, whereby the ansamycine is most preferably rifampicin.

A particularly preferred embodiment is one wherein the combination therapy comprises orally administering RIMSTAR (HRZE) at an individual dose of 5 mg rifampicin/mL; 2.5 mg Isoniazid/ml; 13.3 mg pyrazinamide; 9.2 mg ethambutol/ml). Those skilled in the art will appreciate the standard duration and dosage of these chemotherapy in drug sensitive TB (for example antibiotic treatment, see also Examples 9 and 10).

In combination therapy or adjunctive therapy, administration of these two (or more) substances can be simultaneous, or it can be done in two (or more) inoculations separated over time.

### Materials and Methods

### Reference Materials

a) Monoclonal antibodies: Specific monoclonal antibodies (anti-HSP70, anti-PSTS1, anti-HSP-16.3, (from Lionex Diagnostic GmbH, Braunschweig, Germany)) are used for the identification of the protein profile of FCMtb batches.
b) Albumin Standard: This standard, used for the determination of protein content, is composed of bovine albumin in 0.9% of saline solution (2 mg/ml), conserved in sodium azide (manufacturer: Pierce).
c) Trehalose 6,6′-dimicolate from Mycobacterium tuberculosis (TDM) standard: Commercially available TDM (Sigma) is used for the identification of TDM of FCMtb batches.
d) Mycolic acids from Mycobacterium tuberculosis standard: A commercially available mycolic acid (Sigma) is used for the identification of mycolic acid of FCMtb batches.
e) Molecular weight marker: A commercially available molecular weight marker named SeeBlue Plus pre-stained Standard (Invitrogen) is used.

### Determination of Parameters

a) pH

The pH of the reconstituted suspension of the drug substance (FCMtb) (20 mg/ml) was determined by potentiometry according to Ph.Eur. 2.2.3 and USP<791>.

### b) Water Content

The test for the determination of residual water of the lyophilized FCMtb is carried out using Coulometric Karl Fisher equipment, and it follows the general indications of the Ph. Eur., method 2.5.12, and USP <921> Water determination.

### c) Determination of Total Protein Content

The test for the determination of total protein content of the FCMtb is carried out using a commercial kit (BCA kit, Pierce) and following Ph.Eur., method 2.5.33, method 4 (Bicinchoninic acid or BCA assay) and USP <1057>.

### d) Identification of Protein Profile by Sodium Dodecyl Sulphate Polyacrylamide Gel Electrophoresis (SDS-PAGE)

The test is performed according to Ph. Eur., method 2.2.31 and USP <726>; the detection of proteins in the gel is performed by an adapted Coomassie staining or by Silver staining. Test samples: Reconstitute FCMtb in purified water at 40 or 20 mg/ml concentration. Reference solutions: Molecular weight marker, Purified antigens, FCMtb reference standard.

TABLE 1
Reference antigens
Purified Antigens

M. tuberculosis HSP70 protein (Rv 0350)
M. tuberculosis PsTS1 protein (Rv0934)
M. tuberculosis Ag85 complex (Ag85 B and Ag85B) proteins
(Rv1886c and Rv 3804c)
M. tuberculosis HSP-16.3 protein (Rv2031c)
M. tuberculosis 19 kDa protein (Rv 3763)
M. tuberculosis CFP10 protein (Rv3874)
M. tuberculosis ESAT6 protein (Rv3875)

For Coomassie staining, Gel-Code Blue Stain reagent solution (Pierce) is used according to the manufacturer's instructions. For Silver Staining, the PROTSIL1 Kit from Invitrogen is used according to the manufacturer's instructions. For Western Blot analysis, proteins are separated by SDS PAGE according to standard methods known in the art and then electrophoretically transferred onto a PVDF membrane for immunodetection using specific monoclonal antibodies. The interaction antigen-antibody is visualized by incubation with an anti-antibody which triggers a chemiluminescent reaction. Antigens from Lionex (Braunschweig, Germany) (M. tuberculosis HSP70 protein (70 kDa), M. tuberculosis PsTS1 protein (38 kDa), M. tuberculosis 85 complex (30-34 kDa), and specific monoclonal antibodies anti-HSP70, anti-PsTS1, anti-HSP-16.3, and anti-Ag85B from Lionex are used.

### e) Identification of Mycolic Acids

Mycolic acids in FCMtb are examined by one-dimensional TLC, following the Ph. Eur., method 2.2.27. Test samples: Lyophilised FCMtb, 20 mg. Reference solutions: Mycolic acid standard (Sigma). Procedure:
a) Extraction process: The sample is extracted with chloroform:methanol (1:1) and then it is incubated overnight. The supernatant fraction is eliminated.
b) Mycolic acid esterification: 2 ml of methanol:toluene:sulphuric acid (30:15:1; vol/vol) is added on each tube, and esterification is achieved overnight. Then, 2 mL of n-hexane is added. Recover the supernatant in a new tube, and add 2 ml of n-hexane again. It is dried under nitrogen flow and it is resuspended with 500 μL hexane
c) TLC: 20 μL of each sample is applied on a line parallel to the edge of the plate (Silica gel 60 (20×20 cm) (Merck). The chromatographic separation is performed in a saturated tank with a mobile phase (ethylic ether:n-hexane (15:85, vol/vol)) by three times. Then, the plate is allowed to dry in air.
d) Mycolic acids are revealed by spraying the plates with a solution of phosphomolybdic acid in 96° ethanol and heat at 120° C. for 10 min.

Mycolic acids in FCMtb samples are determined by comparison with mycolic acid commercial standard spot. Results are expressed as qualitative data (presence (positive)/absence (negative) of mycolic acid assessed.

### f) Identification of Trehalose 6,6′-Dimycolate (TDM):

Test samples: Lyophilised FCMtb, 20-40 mg. Reference solutions: TDM standard (Sigma). Procedures:
(i) Extraction process: The sample is extracted with chloroform:methanol (1:1; vol/vol) and then it is incubated overnight. The supernatant fraction is dried under nitrogen flow and it is weighted. Finally, dried samples are resuspended in chloroform at 40 mg/ml final concentration.
(ii) TLC: 10 ml of each sample is applied on a line parallel to the edge of the plate (Silica gel 60 (20×20 cm) (Merck). The chromatographic separation is performed in a saturated tank with a mobile phase (chloroform:methanol:water (60:12:1; vol/vol). Then, the plate is allowed to dry in air.
(iii) Detection: The TDM is revealed by spraying the plates with a solution of antrone 1% in sulphuric acid and heat at 120° C. for 5 minutes.
(iv) Identification: TDM in FCMtb samples is determined by comparison with commercial TDM which is used to generate a standard spot. Results are expressed as qualitative data, i.e. presence (positive)/absence (negative) of TDM assessed.

### g) Identification of Lipoarabinomannan (LAM):

For Western Blot analysis of LAM, compounds are separated by SDS PAGE according to standard methods and then electrophoretically transferred onto a nitrocellulose membrane for immunodetection using specific antibody CS35. The interaction antigen-antibody is visualized by incubation with an anti-antibody (IgG Goat anti-mouse IR Dye 800 CW) which triggers phosphatase alkaline reaction.

### h) Sterility

All processes which require sterility, according to the knowledge of the person skilled in the art, are carried out under sterile conditions; this also applies if sterility is not explicitly mentioned for any given step which requires the same. Sterility test is assessed as prescribed in Ph. Eur. 2.6.1 (USP <71>).

### i) Mycobacteria Inactivation

The inactivation of mycobacteria is assessed in accordance with Ph. Eur. 2.6.2

### j) Bacterial Endotoxins

The test for bacterial endotoxins (LAL test, Limulus Amoebocyte Lysate) is performed according to the general indications of the Ph. Eur., method 2.6.14, following Method D (Chromogenic kinetic method) as well as USP <85>.

### Fragmentation of Bacilli

The choice of fragmentation of the bacilli is thought to allow optimal presentation of cell antigens, particularly cell wall antigens. Fragmentation of the FCMtb (drug substance) is determined by both Dynamic Light Scattering (as described below and Laser diffraction methodologies. Laser diffraction allows measuring the fragmentation in a range between 0.04 μm and 2000 μm. The assay is carried out in the Servicios Científico-Técnicos de la Universitat de Barcelona, Spain. Instrument: Coulter LS 13320 equipped with a Universal Liquide Module (ULM). Solvent: purified water/mineral oil. Results: plotted as a histogram, expressing the relative frequency of the number of particles (%) in front of the particle diameter (0.04 μm-2000 μm).

Determination of z-Average Particle Size and Polydispersity Index

The average particle size as described in this document is determined by dynamic light scattering (DLS), which is based on the physical concept of the Brownian motion of particles, defined in the Stokes-Einstein equation:

$$d ( H ) = \frac{kT}{3 πη D}$$

where:
d (H)=hydrodynamic diameter
D=translational diffusion coefficient
k=Boltzmann's constant
T=absolute temperature
η=viscosity

Without wishing to be limited to any particular theory, the Stokes-Einstein equation establishes that particles suspended in a liquid medium are in a constant and random movement, with a speed that depends on their size: the larger the particle is, the slower the Brownian motion will be.

In dynamic light scattering measurements, the sample containing the particles to be measured is illuminated with a monochromatic light source, preferably a laser, and analyzed in a correlation function how the intensity of scattered light fluctuates with time. If, for instance, large particles are being measured, as they move slowly, the intensity of scattered light fluctuates slowly, and the correlation takes long time to decay; on the other hand, if small particles are being measured, as they move quickly, the intensity of scattered light fluctuates quickly, and the correlation of signal decays more rapidly. According to this invention, the particles are preferably measured with the following instrument: Zetasizer nano zs (Malvern Instruments), using purified water/mineral oil as solvents.

If nothing to the contrary is indicated, the instrument is used according to the manufacturer's instructions, and adjustment and calibration, if applicable, are also according to the manufacturer's instructions.

The size is calculated from the correlation function using various algorithms. In the present case, the ‘cumulants analysis’, as defined in ISO13321 Part 8 is applied. The correlation function fits results in a single exponential curve that allows for calculation of the following parameters:
The mean size, or z-average diameter, of the particle distribution. This mean size is the intensity mean.
The polydispersity index (pdi), i.e. corresponding to the width of the particle size distribution.

The results are typically plotted as a histogram, expressing the relative frequency of the number of particles (%) with respect to the particle diameter which may be any diameter which is comprised in the range from 1 nm to 3 μm.

### Diagnosis of Active Tuberculosis

Methods for diagnosis of active tuberculosis are well known to the skilled person. It is based on the detection of symptoms, like prolonged cough of three or more weeks, chest pain, haemoptysis, etc. Systemic symptoms include low grade fever, chills, night sweats, appetite loss or easy fatiguability. A definitive diagnosis of tuberculosis is based on the detection of Mycobacterium tuberculosis bacilli in the patient samples. The presumption comes from the detection of acid-fast bacilli, which can be confirmed by its culture, or by the detection of M. tuberculosis nucleic acid detection.

### EXAMPLES

The invention is in the following illustrated by examples. The examples are for illustrative purposes and should by no means be understood as limiting the scope of the present invention.

### Example 1: Isolation of the Strain Mycobacterium tuberculosis NCTC 13536

The starting material for the production of FCMtb (drug substance) is an inoculum of the strain NCTC 13536, synonymously called 511 or Mycobacterium tuberculosis NCTC 13536 or Mycobacterium tuberculosis strain RUTI, a strain of Mycobacterium tuberculosis isolated from an immunocompetent patient diagnosed with pulmonary tuberculosis in Barcelona, Spain. It was deposited in 2010 at the NCTC in London, which is an official depositary organisation according to the Budapest Treaty. The strain has additionally been deposited by the strain collection of the Service of Microbiology of the Hospital de Sant Pau, Barcelona, Spain.

Two passages of the original strain have been performed in the years 1995, and 1996 respectively. MSL PB #1 corresponds to the second passage of the original strain of M. tuberculosis NCTC 13536, which was performed in October 1996 resulting in 100 vials (3 ml sterile glass vials) stored at-70±5° C. The strain has a low genetic polymorphism, as identified by standard methods in the art.

### Example 2: Upstream Process for Production of the Drug Substance: Production of MTB-C Cells

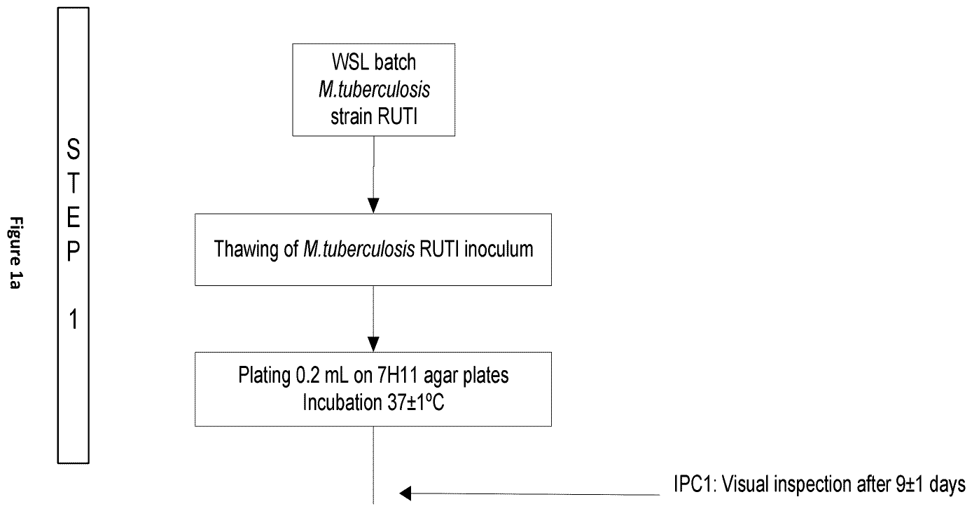

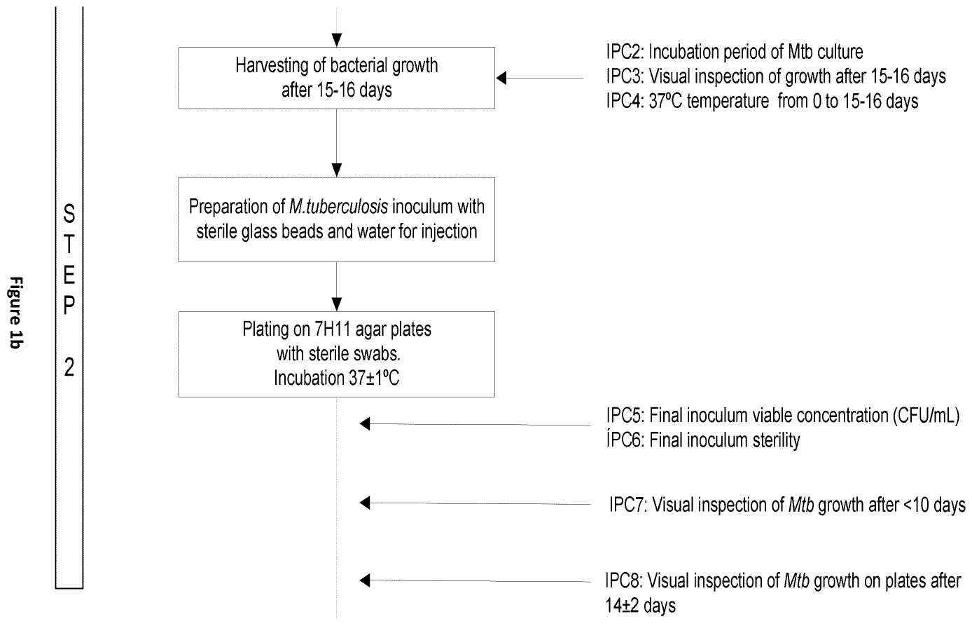

A flow chart of this process is given in FIG. 1. The starting material for the production of FCMtb (drug substance) is an inoculum of the strain Mycobacterium tuberculosis NCTC 13536 (Example 1). In order to ensure the continued supply of this starting material, a seed lot system is preferably used. Hence, a working seed lot (WSL) derived from a master seed lot (MSL) is used for production of FCMtb.

TABLE 2
current in-process controls (IPCs) performed in the upstream process

| Step of | | | | | |
|---|---|---|---|---|---|
| the | IPC | Parameter | | Acceptance | |
| process | number | controlled | Test | criteria | Objective |
| Step 1 | IPC1 | Visual inspection of | Visual | Thin layer of Mtb | To check growth and absence of |
| | | Mtb culture after | inspection | culture | contamination |
| | | 9 ± 1 days | | Absence of | |
| | | | | contamination | |
| Step 2 | IPC2 | Incubation period of | Time | 15-16 days | To check that the incubation |
| | | Mtb culture | observation | | period has been achieved |
| | IPC3 | Visual inspection of | Visual | Ivory colour, | To check the correct colour and |
| | | growth after 15-16 | inspection | rough appearance | appearance of Mtb culture and |
| | | days | | | absence of contamination |
| | IPC4 | 37° C. temperature | Temperature | 37 ± 1° C. | Maintenance of the 37° C. ± 1° C. |
| | | from 0 to 15-16 | control | | temperature conditions during the |
| | | days | | | whole incubation period to ensure |
| | | | | | appropriate growth |
| | IPC5 | Final inoculum | Viable plate | 0.75E+08 − | To control the concentration of |
| | | viable | count | 3.00E+08 | Mtb viable of final inoculum after |
| | | concentration | | CFU/mL | seeding on 7H11 plates |
| | | (CFU/mL) | | | |
| | IPC6 | Final inoculum | Sterility (Ph. | Sterile | To ensure the absence of bacterial |
| | | sterility | Eur. 2.6.1) | | and fungal contamination in the |
| | | | | | Mtb inoculum prior to and during |
| | | | | | seeding on 7H11 plates |
| | IPC7 | Visual inspection | Visual | Presence of Mtb | To ensure the presence of Mtb |
| | | of Mtb culture | inspection | culture | culture |
| | | after <10 days | | | |
| | IPC8 | Visual inspection of | Visual | Ivory colour | To ensure the presence of Mtb |
| | | Mtb culture on | inspection | | culture and absence of |
| | | plates after 14 ± 2 | | | contamination by visual inspection |
| | | days | | | |
| Step 3 | IPC9 | 37° C. from 0 to 21 ± 1 | Temperature | 37 ± 1° C. | To ensure that 37° C. temperature |
| | | days | control | | conditions have been maintained |
| | | | | | throughout the whole incubation |
| | | | | | period until harvest |
| | IPC10 | Visual inspection of | Visual | Confluent | To control appropriate Mtb |
| | | mycobacterial | inspection | appearance | culture and absence of |
| | | growth on plates | | Ivory colour | contamination |
| | | after 21 ± 1 days | | Absence of | |
| | | | | contamination | |
| | IPC11 | Sterility of the | Sterility (Ph. | Sterile | To ensure the absence of bacterial |
| | | HMtb | Eur. 2.6.1) | | and fungal contamination in the |
| | | | | | HMtb prior to freezing |

### (1) Culture of Mtb WSL

The production of the DS begins with the thawing of one vial of WSL batch. Each WSL vial contains 0.5 mL and is used to produce two WSL cultures that are run in parallel. To that purpose, for each WSL culture, 0.2 mL of the WSL are seeded on 7H11 agar plates and incubated at 37±1° C. for 15-16 days. A visual inspection of the growth and absence of contamination is performed after 9±1 days of incubation (IPC1).

### (2) Harvest of Mtb WSL and Expansion of Mtb Culture

After 15-16 days of incubation of Mtb WSL, completeness of incubation period is recorded (IPC2) and a visual inspection of the growth is carried out to check the appearance and colour of bacterial culture (IPC3). The register of temperature is controlled to confirm that the incubation temperature of 37±1° C. has been maintained throughout the whole incubation period (IPC4).

Colonies from the Mtb WSL culture are then transferred into a tube containing a few glass beads and, water for injection is added up to a final concentration of 9-10 mg/ml of Mtb to obtain the Mtb inoculum for culture expansion. After mixing the bacterial suspension, 200-1008 7H11 agar plates are seeded with Mtb inoculum using sterile swabs soaked with the bacterial suspension to obtain confluent cultures. The plates are incubated at 37±1° C. for 21±1 days under atmospheric control. At this point, two IPCs are carried out on the final inoculum suspension (the inoculum after plates seeding): a final inoculum viable concentration (CFU/mL) (IPC5), and final inoculum sterility test (IPC6).

During incubation of seeded plates at 37±1° C., a visual inspection of the growth and absence of contamination is performed before 10 days of incubation (IPC7) and after 14±2 days (IPC8).

### (3) Harvest of Mtb and Freezing

After 21±1 days of incubation at 37±1° C. and before harvesting, a temperature register is checked to confirm that the incubation temperature of 37±1° C. has been maintained throughout the whole incubation period until harvest (IPC9). Afterwards, bacterial growth is collected from agar plates and transferred into sterile tubes to obtain the Harvest of Mtb (HMtb). A visual inspection is performed to check appropriate Mtb growth and purity of the bacterial culture while harvesting and the final register is done once harvest has been finished (IPC10). The weight of HMtb should be on the range of 72-400 g. HMtb is then frozen and stored at −80° C.±5° C. Sterility test of the HMtb is carried out on the first plate harvested (IPC11).

### Example 3: Downstream Process for Production of the Drug Substance

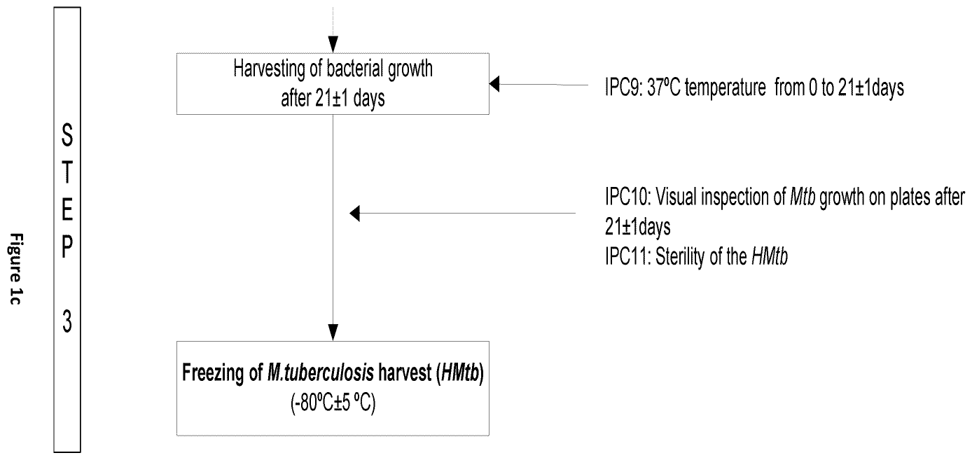

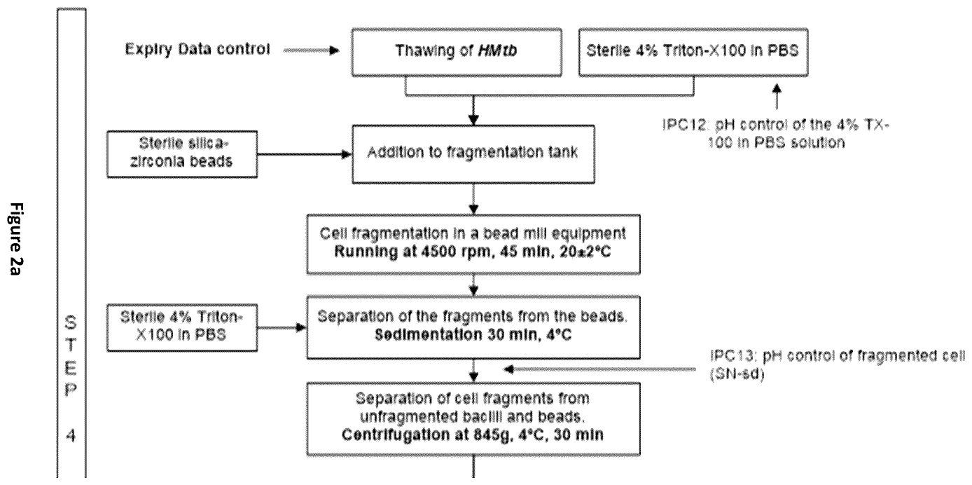

A flow chart of this process is given in FIG. 2.

TABLE 3
current in-process controls (IPCs) performed in the downstream process

| Step of the | | Parameter | | Acceptance | |
|---|---|---|---|---|---|
| process | IPC number | controlled | Test | criteria | Objective |
| Step 4 | IPC12 | pH of the 4% | pH test strip | pH 7.0-7.7 | To ensure the |
| | | TX-100 in PBS | | | reproducibility of the |
| | | solution | | | process |
| | IPC13 | pH control of | pH test strip | pH 6.4-7.0 | To control the |
| | | fragmented | | | successful completion of |
| | | cells (SN-sd) | | | cell fragmentation |
| | IPC14 | Visual | Visual | Clear and | To ensure the efficiency |
| | | inspection of | inspection | Colourless | of centrifugation and |
| | | the SN after | | | PBS washes at |
| | | second high- | | | removing lipid |
| | | speed | | | supernatant |
| | | centrifugation | | | |
| | IPC15 | Sterility of the | Sterility (Ph. | Sterile | To ensure the absence |
| | | PBS | Eur. 2.6.1) | | of bacterial and fungal |
| | | | | | contamination |
| Step 5 | IPC16 | Cell viability of | Viable count | <1.0E+09 | To ensure cell viability |
| | | FCMtb | plate | CFU/totals < | before pasteurisation |
| | | suspension | | 80 ml | |
| | | (CFU/mL) | | | |
| Step 6 | IPC17 | FCMtb | Sterility (Ph. | Sterile | To ensure the sterility of |
| | | suspension | Eur. 2.6.1) | | the suspension after |
| | | sterility | | | pasteurisation |

### (4) Cell Fragmentation and Delipidation (Purification)

The frozen HMtb (Example 2) is thawed by means of a thawing slope (STEP 1: 10 h at 10° C. (0.1° C./min); STEP 2: 6 h at 4° C. (0.1° C./min) and STEP 3: oh at 8° C. (0.1° C./min). Sterile PBS buffer with 4% triton-X100 (pH 7.0-7.7, see IPC12) is then added and subsequently transferred into a sterile fragmentation tank containing sterile silica-zirconia beads. Then, cell fragmentation is carried out in bead mill equipment at 4500 rpm for 45 min at 20±2° C.

Once the process is finished, the cellular fragmented fraction is separated from the beads by subsequent washings (repeated shaking and sedimentation cycles) in sterile PBS buffer with 4% triton-X100 (pH 7.0-7.7). At this point, a pH control of the cell fragments (supernatant) is carried out (IPC13). Afterwards, a final centrifugation at 845 g at 4° C. for 30-45 minutes is performed to separate cell fragments from whole bacilli and beads. The supernatant is harvested.

To remove cytosolic fraction and get a suspension enriched in cellular fragments, the supernatant is centrifuged twice at high speed (27.000 g) approx. for 60 minutes, at 4° C. After the first centrifugation the yellowish supernatant (rich in soluble proteins and lipids) is discarded and the pellet is resuspended in PBS and further centrifuged in the same above described conditions. After that the appearance of the discarded supernatant (IPC14) must be clear and colourless. The obtained pellet is weighed (>10 g) and resuspended with an appropriate volume of sterile PBS (45-150 mL) to obtain a maximum concentration of 0.2 mg/mL (DS or FCMtb suspension).

Besides, the sterility of the PBS used for the purification of cell fragments and pellet resuspension is tested (IPC15).

### (5) Pasteurization

In order to inactivate the residual bacilli, the whole bulk of DS, FCMtb (drug substance) suspension, is pasteurised at 65±2° C. for 60-65 min. However, before pasteurisation, an aliquot of FCMtb suspension is taken out for testing cell viability (CFU/mL) (IPC16). Once material is subjected to pasteurisation, it will be physically segregated from untreated material, i.e., spaces used up to pasteurisation are clearly separated from those used during the subsequent filling process.

### (6) Filtration and Filling3

After pasteurisation, the bulk of DS leaves from the BSL3 room to enter to a positive sterile room (B/A-1 class). Elimination of residual beads is performed through filtration (40 μm), and sterile and depyrogenated vials are filled with 0.5-2 ml of the FCMtb suspension. Four filled vials are used for sterility testing (IPC17). Finally, filled vials are freeze-dried

### (7) Freeze-Drying, Encapsulation and Labelling

All vials are lyophilised at about −45° C. to 30° C. temperature and at 0.310 mbar pressure for approximately 18 hours (0.5 ml volume per vial) and under N₂ atmosphere.

Once lyophilised, vials are encapsulated and labelled and capped using aseptic techniques. This is a continuous process: filled vials are visually inspected and if they are correct (good appearance of the cake, correct dose, non-broken vials), they are first capped with an aluminium cap and subsequently labelled (if visual inspection of capped vial is correct) as follows:
number of vial,
name of the product,
batch code,
manufacturing date
storage condition
shelf-life.

Overall, vials are visually inspected after freeze-drying, during capping and during labelling. The packaged DS is then stored at −20° C.±5° C.

### Example 4: Protein Characterisation of the Drug Substance

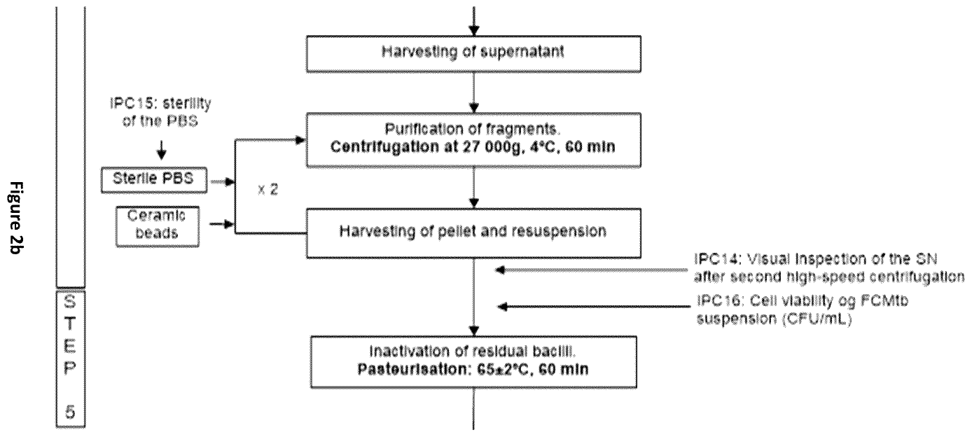

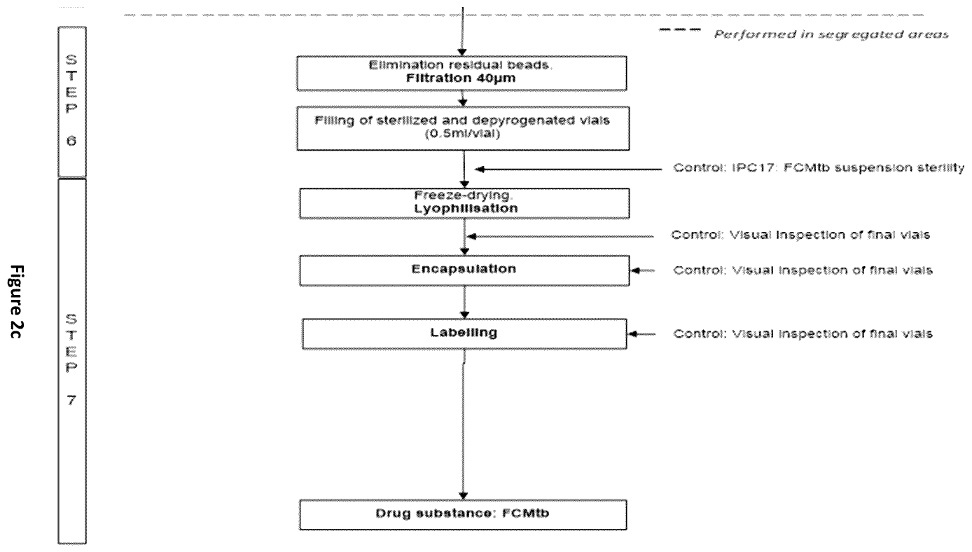

Based on literature (Andersen P., 1997, Sc and J. Immunol.; 45(2):115-31; Geisel et al., 2005, J. Immunol.; 174(8):5007-15; Stewart et al. 2005, Infect. Immun., 73(10):6831-7., Wang et al., 2007, J. Mol. Biol., 366(2):375-81 Rodriguez-Hernandez, et al. 2020, Biomed and Biotechnol 21(11):856-870; Meier et al., 2018, Frontiers in Immunology Vol 9 Article 2476), some protein bands were selected as being representative for protein profile assessment: Heat shock protein (HSP) 70 protein (Rv0350); Phosphate binding (PsTS1) (38 kDa) protein (Rv 0934); outer cell wall antigen 85 complex (30-34 kDa) (Rv3804c & Rv1866c); Heat shock HSP16 (16 Kda) protein (Rv 2031c); 19 kDa protein (Rv 3763), CFP10 protein (Rv3874), and ESAT-6 protein (Rv3875).
(A) Determination of total protein content: Total protein levels in FCMtb are quantified by bicinchoninic acid (BCA) methodology. Total protein represents about 15% (w/w) of FCMtb content. Reference standards FCMtb-81, FCMtb-83, FCMtb-86 and FCMtb-87 contain 189, 187, 150 and 160 μg protein/mg FCMtb, respectively.
(B) Identification of protein profile by sodium dodecyl sulphate polyacrylamide gel electrophoresis (SDS-PAGE): The protein profile of the drug substance FCMtb was determined by comparison with reference antigens from Mycobacterium tuberculosis corresponding to ESAT6 (6 kDa) (7); CFP10 (10 kDa) (6); HSP16 (16 kDa), Ag85 complex (30-34 kDa) (1); PsTS1 (38 kDa) (2); HSP70 (70 kDa) (4) as well as Molecular Weight marker MW (5), are shown (see FIG. 3). Determination of protein profile of drug substance FCMtb and identification of bands (approximately 70 kDa, 38 kDa, 30-34 kDa and 16 kDa is carried out by Coomassie staining. Identification of the 19 kDa (lipopolypeptide), 10 kDa, and 6 kDa band is carried out by silver staining.
C. Identification of the protein profile by Western-blot with specific monoclonal antibodies: The protein profile of the drug substance FCMtb is determined by the patterns obtained using Western-blot analyses with monoclonal antibodies (mAb): Anti-HSP70 (70 kDa), anti-PsTS1 (38 kDa), anti-Ag85B (85 complex 30-34 kD) and Anti-HSP16.3 (16 kDa) (see FIG. 3c).

FCMtb-81 is the reference batch for FCMtb (drug substance) according to the preferred mode of carrying out this invention.

### Example 5: Lipid Characterisation of the Drug Substance

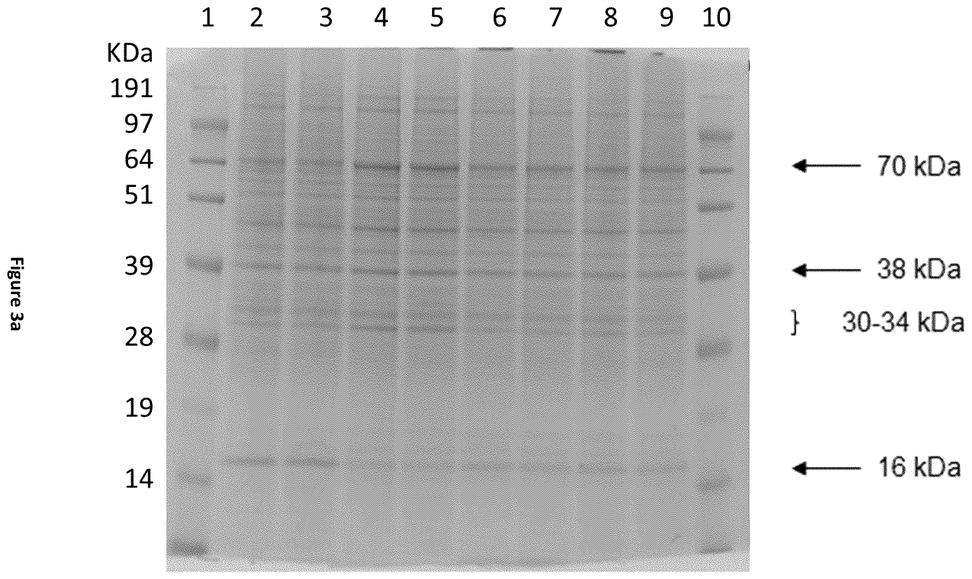

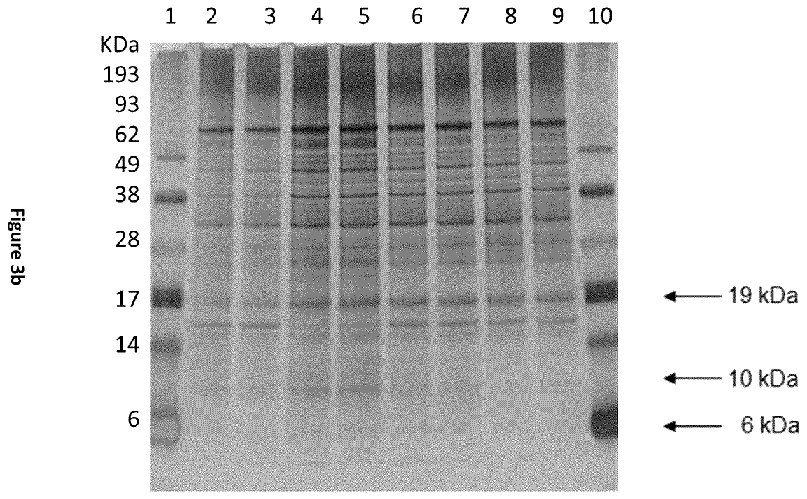

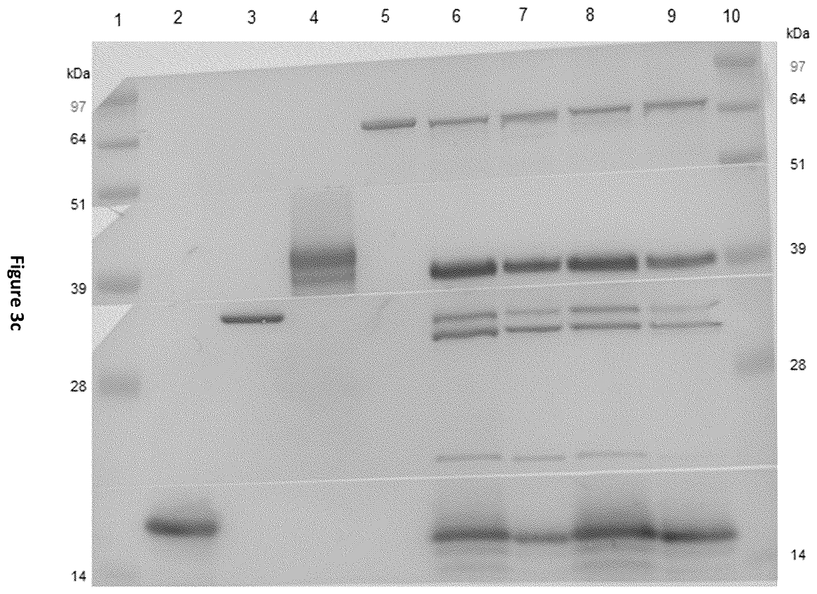

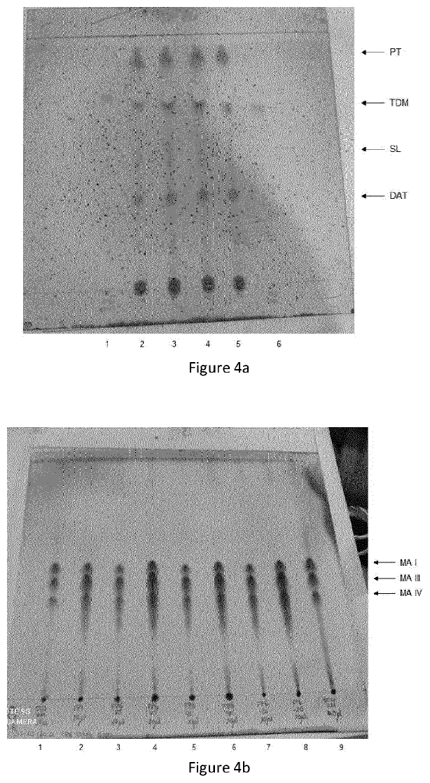

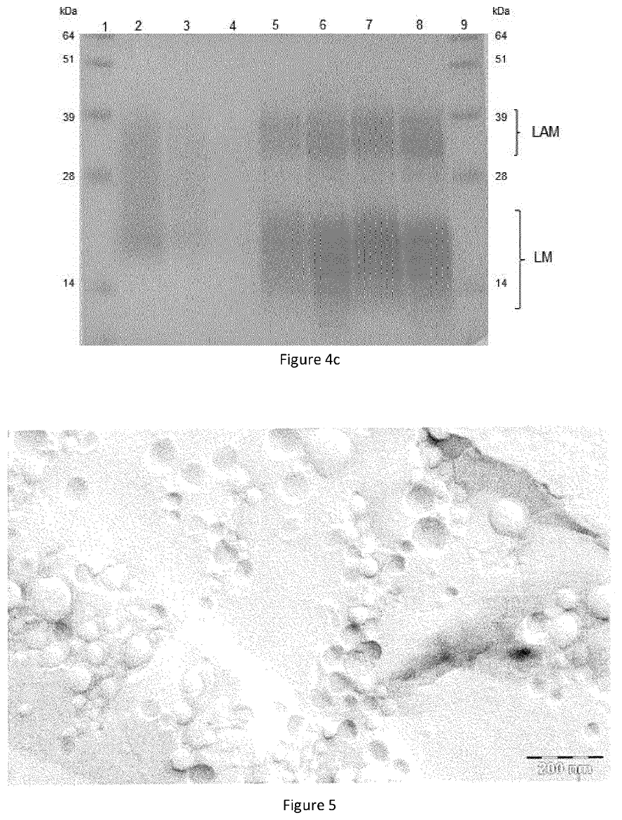

The characterisation of the lipid profile of FCMtb (drug substance) consists of a fractionating process based on chloroform:methanol (1:1) extraction. The fractionating process carried out in these studies has been based on the procedure described by Delmas et al., 1997, Glycobiology 7(6), 811-7. Thin layer chromatography (TLC) has been the method used to analyse the content of lipids and glycolipids present in FCMtb. Specifically, polyaciltrehalose (PT), trehalose dimycolate (TDM), diacyltrehalose (DAT), sulfolipids (SF) and other phospholipids, as well as Mycolic acids have been identified by thin layer chromatography (TLC) both in in different FCMtb batches. See FIG. 4a. The content of trehalose 6,6′-dimycolate (TDM) is analysed by TLC in the supernatant. Mycolic acids determination is conducted in the sediment, following a TLC method. Although no quantitative data are known for the lipid profile of MTB-C, the qualitative lipid profile established in the studies is in line with current scientific knowledge and allows for a standard characterisation of the immunogenic lipids so far known. Overall, the lipid content has been shown to be consistent in different batches of FCMtb-comprising liposomes according to this invention. FIG. 4c shows the identification of LAM and LM.

### Example 6: Characterisation of Fragmented Cell Material

Preliminary results using both methodologies show that the fragments size of FCMtb (drug substance) is mainly below 1 μm (99%<1 μm) which is corroborated by FCMtb electronic microscopy: the fragment size ranges mainly from 100 to 300 nm.

Levels of residual DNA after extraction with a phenol/chloroform mixture have been investigated by absorbance at 260 nm (detection limit: 0.2 μg DNA/mg FCMtb). Typical results obtained so far are below 10 μg DNA/mg FCMtb.

The consistency of the production process of the drug substance is shown by a lipid and protein profile that is reproducible for different FCMtb batches.

### Example 7: Effect of Sucrose

In an initial test, one of the following excipients (a) 1.5% glycine and (b) 5% sucrose, respectively, was optionally incorporated into a liposome formulation comprising fragments of the MTB-C strain NCTC 13536. Subsequently, a comparative evaluation is undertaken on physicochemical properties and biological activity associated to both formulations.

Results obtained by measurement after reconstitution of the lyophilized liposome composition and after testing according to the current batch release specification parameters are presented in Table 4.

TABLE 4
Results of specifications for 3 different formulations, measured
after reconstitution of the lyophilized liposome composition.

| | | | FCMtb formulated in a | | |
|---|---|---|---|---|---|
| | | | liposome suspension | | |
| | | | Without | Sucrose | Glycine |
| | | | excipient | 5% | 1.5% |
| | | | FCMtb formulated in a | | |
| Parameter | Acceptance criteria | | liposomal suspension | | |
| Appearance | White to off-white powder | | ND | Complies | Complies |
| Cake morphology | Flat to almost flat and | | ND | Complies | Complies |
| | homogeneous | | | | |
| Water content (%) | ≤3% | | ND | 1.4 | 3.0 |
| Time to reconstitution (s) | Well reconstituted ≤10 s | | ND | 5 | 12 |
| pH | 7-8 | | 8.1 | 7.4 | 7.0 |
| Particle size | 75 ± 20 (≤0.250) | | 370⁽¹⁾ | 66 | 504⁽¹⁾ |
| z-average (nm) Pdi (a) | | | (0.492) | (0.215) | (0.569) |
| Immunogenic potency in | PPD | 3-12 Ratio SFU/106 | 3.6 | 5.1 | 2.8 |
| M. Tuberculosis | | cells with respect | (183) | (183) | n |
| infected murine model | | to basal value | | | |
| Antigen-specific IFN-γ | | (Basal value in | | | |
| Spot forming units | | SFU/106 cells) | | | |
| | Ag85B | 5-20 Ratio SFU/106 | 5.5 | 9.6 | 5.4 |
| | | cells with respect | (73) | (73) | (73) |
| | | to basal value | | | |
| | | (Basal value in | | | |
| | | SFU/106 cells) | | | |

(a) Polydispersity index
⁽¹⁾Presence of liposomal aggregates
ND = not determined

The investigators of this study surprisingly found that 5% sucrose formulation provides the advantage of better physicochemical results, such as water content or time to reconstitution, respectively. But the most crucial fact is the important reduction in liposomal aggregation (particle size, z-average) shown with the sucrose-comprising formulation in comparison with the other two formulations. Analysis by Dynamic Light Scattering has shown a z-average of 75±20 nm (polydispersity index ≤0.350) of the sucrose-containing liposomes. Electron microscopy of freeze-fracturing preparations of the sucrose-containing liposome formulation shows a mixture of multilamellar and unilamellar liposomes with sizes between 40 and 100 nm (FIG. 5).

Due to this improved parameter together with the observed lesser water content levels (≤2%) for the 5% sucrose formulation, improved stability results are expected for this formulation. This is indeed the case.

### Example 8: Manufacturing Process of Lyophilised Liposome Formulation (Drug Product)

One embodiment of the manufacturing process of a pharmaceutical composition comprising the liposome formulation according to the present invention is shown in FIG. 6.

The manufacturing process of the drug product, most preferably RUTI, can be divided into the following main steps:
Step 1: Preparation of the LCS bulk components
Step 2: Preparation of LCS bulk
Step 3: Dilution of LCS bulk to obtain the LS bulk and final sterilization by filtration
Step 4: Filling
Step 5: Lyophilisation, encapsulation, labelling and packaging

### (1) Preparation of the LCS Bulk Components

The lipid phase of the liposomes consists of soy lecithin solution, sodium cholate solution, and the DS (FCMtb, see examples 1-7). Soy lecithin is dissolved in Ethanol absolute GR (1:1; w/w) and sodium cholate is dissolved in WFI (1:5; w/w). The solutions are sterilized by filtration through 0.2 μm membrane filters. The lipid phase is prepared in a class C room.

The aqueous phase consists of 0.9% sterile saline solution (NaCl) diluted with sterile WFI to get a 0.34% final saline solution. This saline solution is prepared in a clean Class A/B room.

### (2) LCS Bulk Preparation

For the preparation of the LCS bulk is necessary mix of sodium lecithin solution and sodium cholate solution; afterwards lyophilised FCMtb is added upon stirring. The ratios of the components are 0.03:0.2:0.7 (FCMtb (drug substance):sodium cholate:soy lecithin; w/w/w).

The aqueous phase is transferred to a sterilised stainless-steel mixer. The lipid phase (containing soy lecithin, sodium cholate, and DS FCMtb) is then added in a ratio 2.7:1 (aqueous phase:lipid phase, w/w). The phases are mixed at 22000 rpm for 3 minutes for homogenisation and liposome formation.

After homogenisation, the bulk LCS is transferred to another vessel and allowed to stand for at least 5 minutes. An in-process Control on particle size is performed on the LCS bulk.

### (3) Dilution of LCS Bulk to Obtain the LS Bulk and Sterilization by Filtration

A 6.0% (w/w) solution of sucrose is prepared with WFI and sterilized by filtration (0.2 μm membrane filter). The sucrose solution is then mixed with sterile WFI and LCS bulk in the adequate proportions to get the final LS bulk constituted of 10.5 mg LCS/mL in 5% sucrose solution (1.2 L), which is sterilized by filtration (0.2 μm membrane filter).

Before of use of the filter and after filtration controls for filter integrity are performed. PCs are carried out before and after sterilized filtration: visual inspection (IPC2), contamination test (IPC 3), particle size (IPC4) and sterility (IPC5).

### (4) Filling

Vials are filled with 0.4 mL of LS bulk (under continuous agitation) and partially closed for lyophilisation.

### (5) Lyophilisation, Packaging, and Labelling

The lyophilisation process is performed in the range of −45° C. to 25° C. temperature and 0.150 mbars. The process lasts for 27 hours.

At the end of lyophilisation vials are fully stoppered in N₂ atmosphere. Then the vials are encapsulated, labelled and stored at 5° C.±3° C. Visual inspections, in each vial, are performed throughout this process, after lyophilisation, after encapsulation and after labelling.

The label contains the following information:
Number of vial
Name of the product
Batch code
Manufacturing date
Storage condition
Shelf life
“Experimental use”.

For the clinical batch, the label contains the requirements stated in GMPs.

### Example 9: Efficacy of Liposome Formulations (Drug Product) Comprising Fragments from a Mycobacterium tuberculosis-Complex (Drug Substance, FCMtb) in Active TB C3Heb/FeJ Mice Model, 200 μg Dose

The liposome formulations (drug product, Example 8) comprising fragments from a Mycobacterium tuberculosis-complex (drug substance, Examples 1-7) used in this example is the most preferred embodiment: RUTI. It is labelled correspondingly in the following examples, including all associated figures. The dosage refers to the dosage of the drug substance, FCMtb.

### Objective

The aim of these experiments was to evaluate the efficacy of the liposome formulations (drug product, RUTI) comprising fragments from a Mycobacterium tuberculosis-complex (drug substance, FCMtb) in a C3Heb/FeJ model of active M. tuberculosis infection as reduction of bacillary load; and improvement of lung damage and T cellular immunoresponse under chemotherapy (RIMSTAR, HRZE).

### Experimental Design

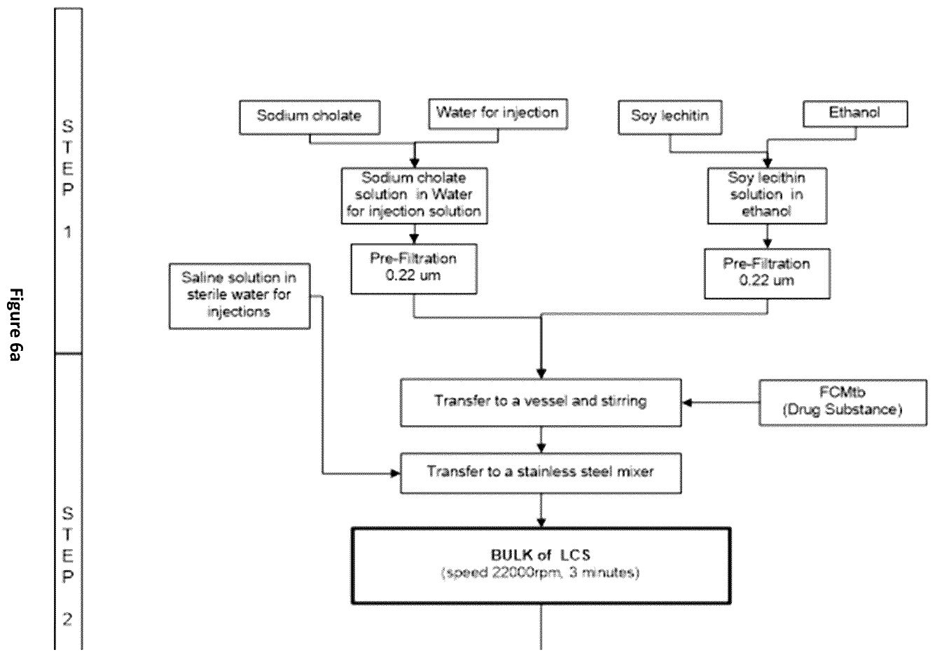

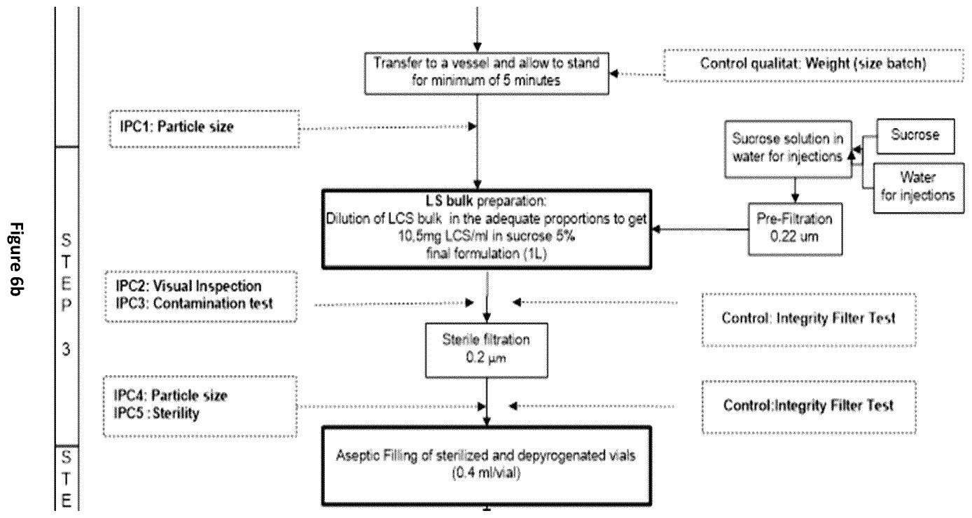

The study was conducted to assess the role of the liposome formulations comprising fragments from a Mycobacterium tuberculosis-complex in the reduction of bacillary load, the improvement of lung damage and cellular immunologic response in Mycobacterium tuberculosis infected mouse strain (C3HeB/FeJ) under chemotherapy (RIMSTAR, HRZE). The liposome formulation was administered as a single dose of 200 μg FCMtb at 0, 1, 2 and 3 weeks after starting the chemotherapy treatment and the sacrifice was done, for example, starting at 1 week after RUTI vaccination (Table 5 and FIG. 7).

TABLE 5
timings of Example 9, ATB antibiotic (HRZE).

| | | Day/Date/Action/Animals number | | | | | | | |
|---|---|---|---|---|---|---|---|---|---|
| | | | | | w 1 | w 2 | w 3 | | |
| | | | | | after | after | after | | |
| Study number: | | | | | first | first | first | w 4 after first | |
| R822.Group | N | w 0 | d 25 | | ATB | ATB | ATB | ATB and/or RUTI | |
| description | 60 | 20-Dec | 14-Jan | | 21-Jan | 28-Jan | 04-Feb | 07-Feb | 11-Feb |
| R822 TB infection | 5 | Infection | sac 5 | — | — | — | — | — | — |
| control | | | | | | | | | |
| R823 Control | 20 | | First | — | sac 5 | sac 5 | sac 5 | final | sac 5 |
| Tt ATB (HRZE) | | | HRZE | | (ATB | (ATB | (ATB | HRZE | (FINAL |
| | | | | | w 1) | w 2) | w 3) | | ATB w 4) |
| R824 Tt HRZE + | 10 | | First | RUTI | sac 5 | — | — | final | sac 5 |
| RUTI w 0 after | | | HRZE | | (RUTI | | | HRZE | (FINAL |
| starting ATB | | | | | w 1) | | | | RUTI w 1) |
| R825 Tt HRZE + | 10 | | First | — | RUTI | sac 5 | — | final | sac 5 |
| RUTI w 1 after | | | HRZE | | | (RUTI | | HRZE | (FINAL |
| starting ATB | | | | | | w 2) | | | RUTI w 2) |
| R826 Tt HRZE + | 10 | | First | — | — | RUTI | sac 5 | final | sac 5 |
| RUTI w 2 after | | | HRZE | | | | (RUTI | HRZE | (FINAL |
| starting ATB | | | | | | | w 3) | | RUTI w 3) |
| R827 Tt HRZE + | 5 | | First | — | — | — | RUTI | final | sac 5 |
| RUTI w 3 after | | | HRZE | | | | | HRZE | (FINAL |
| starting ATB | | | | | | | | | RUTI w 4) |

The results presented are from a single study, using 5 females C3HeB/FeJ mice per group. Mice were infected intravenously with 1.0E+04 of M. tuberculosis H37Rv Pasteur strain (Batch 10 from UTE stock IGTP) via caudal vein. RIMSTAR (HRZE) chemotherapy administration was orally administered a dose of 0.2 ml (5 mg rifampicine/mL; 2.5 mg Isoniacine/ml; 13.3 mg pyraminazide; 9.2 mg ethambutol/ml).

Groups RUTI/sham (saline control) were administrated subcutaneously at 0, 1, 2 and 3 weeks after starting chemotherapy treatment. RUTI dose was 200 μg of FCMtb in 0.2 ml (see Table 5). Animals were sacrificed at different time points (FIG. 7, Table 5). After the sacrifice, the lung and the spleen were removed in order to assess the bacillary load (CFU), the lung damage and the T cell immunological response (from splenocytes).

The effect of RUTI on the reduction of bacillary load, the improvement of lung damage and the cellular immune response (ELispot) were studied.

All procedures were performed in a BLS3 security facility (CMCiB) according to protocol DMAH6119, which was reviewed by the Animal Experimentation Ethics Committee of the Hospital Universitari Germans Trias i Pujol (registered as B9900005) and approved by the Dept d'Agricultura, Ramaderia, Pesca, Alimentació i Medi Natural of the Catalan Government, according to current national and European Union legislation regarding the protection of experimental animals (Law 1997 of the Catalan Government; Spanish Royal Decree 1201/2005; and European legislation 86/609/EEC; 91/628/EEC; 92/65/EEC and 90/425/EEC).

### Detailed Experimental

### Bacillary Load (CFUs/mL):

Samples of lung lobes from each animal were collected, homogenized and several dilutions plated on nutrient Middlebrook 7H11 agar (BD Diagnostics, USA). The number of CFU was counted after incubation for 21 days at 37° C. and the results expressed as CFU/ml.

### Lung Pathology:

Right lower lung lobe samples were fixed in 10% buffered formalin, embedded in paraffin and 5-μm sections stained with haematoxylin-eosin for microscopic observation and analysis of the damaged area using the NISElements D version 3.0x software package (Nikon Instruments Inc., Tokyo, Japan). Four recuts of a block containing all group samples were used to determine the damaged area as a percentage of total lung area.

### T Cell Immunological Response (Elispot Technique):

Spleen were mechanically disrupted and filtered through a 40 μm cell strained (BD Diagnostic, USA), with erythrocytes being incubated for 8 min in lysis buffer (Tris 17 mM, NH4CL 0.14M). Cell culture was conducted in supplemented RPMI 1640 (10% Fetal Calf Serum, streptomycin 100 g/ml, penicillin 100 U/ml, 2-mercaptoethanol 0.025 mM, sodium pyruvate 2 mM) in 96 well plates (ELISpot plates) at 37° C. and 5% CO₂, with or without stimulus (PPD, ESAT-5, HSP16.3 and PsTS1). ELISPOT assays T cells based on cytokine production. Cytokine secreted by individual activated T cells as discrete spots on a plastic plate, which are counted to give the number of activated T cells. Each well contained 125.000 cells. Final concentration of PPD at 30 μg/ml well and ESAT-6, HSP16.3 and 85B antigen at 10 μg/ml well. The stimulus were from Statem Serum Institute Denmarx (PPD) and from Lionex Diagnostic and Therapeutics, German (ESAT-6, HSP16.3 and 85B antigen). After 24 h of stimulus incubation ELISPOT is carried out to assess the number of spots induced in each condition. ELISPOT reader (IGTP) counted the spots.

### Mtb Cell Wall Fragment Containing Drug Product:

The Drug Substance (DS) consists in purified cell wall fragments of Mtb named FCMtb. The manufacturing process of FCMtb is obtained by cellular fragmentation of harvested bacterial cultures of Mtb strain (see previous examples). Afterward the fragments cell walls are purified in presence of Triton X100 to remove the soluble components and the endotoxin-like molecules. The structure of the DS is tightly related to the characteristics of the cell wall of Mtb. Thus, DS is composed mainly by a heterogeneous spectrum of proteins and lipids from the Mtb strain. RUTI, the IMP (drug product), is the DS liposome suspension with a charge excipient. It is presented as a dry powder for reconstitution with water for injection and it is stable at 5° C. for at least 12 months.

TABLE 6
Quantitative and qualitative composition of RUTI per vial.

| Components | Unit per vial | Function |
|---|---|---|
| FCMtb | 266.7 μg | Immunogen |
| Sucrose | 20,000.0 μg | Charge substance (freeze- |
| | | drying) and cryoprotector |
| Soy lecithin ¹ | 3,383.2 μg | Liposome forming agent |
| Sodium cholate | 368.0 μg | Tensoactive |
| Sodium chloride ² | 83.2 μg | Isotonic agent |
| Ethanol ³ | q.s. | Solvent |
| Water for injection ³ | q.s | Solvent |

¹ Containing Phosphatidylcholine (Not less than [NLT] 94.0%)
² Added as NaCl 0.9% solution
³ It disappears in the course of processing

For administration to mice, the investigational medicinal product (IMP, drug product, RUTI) should be reconstituted with 0.266 ml of water for injection to give a solution containing 1002.1 μg/mL of FCMtb (FCMtb 266.7 μg/vial). The composition of the placebo to be used in the non-clinical trial presented is saline (0.9% NaCl) A total volume of 0.200 ml of reconstituted RUTI vaccine will be administered subcutaneously in the neck area.

### Data Analysis—Statistical Methods:

GraphPad Prism version 8.0 for Windows (GraphPad Software, San Diego California USA) was used for graphics and statistics, with differences of p<0.05 being considered statistically significant. Data are shown as mean for each treatment group. Statistical analysis is performed using unpaired t-test to look for any differences between experimental groups.

### Results

### Bacillary Load in Lung and Spleen Tissue

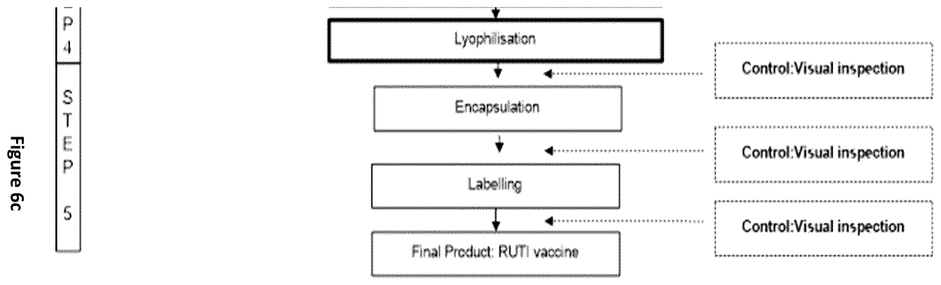

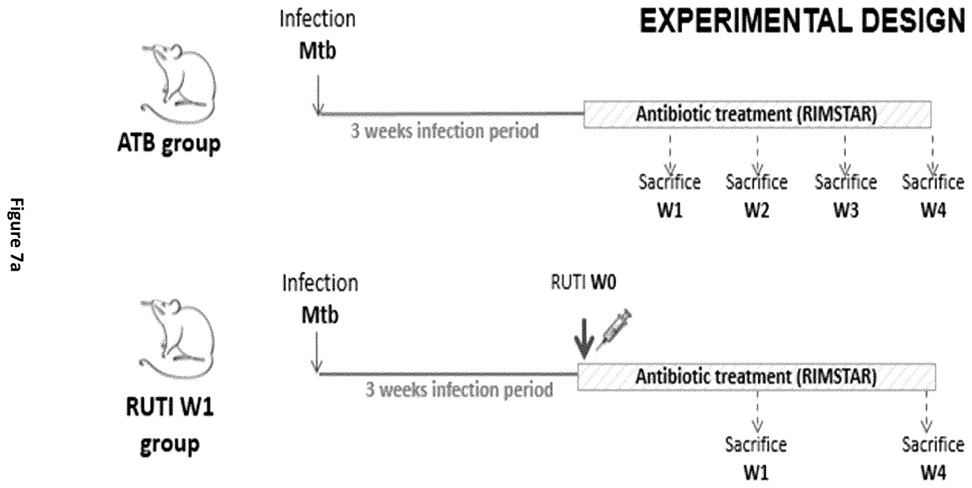

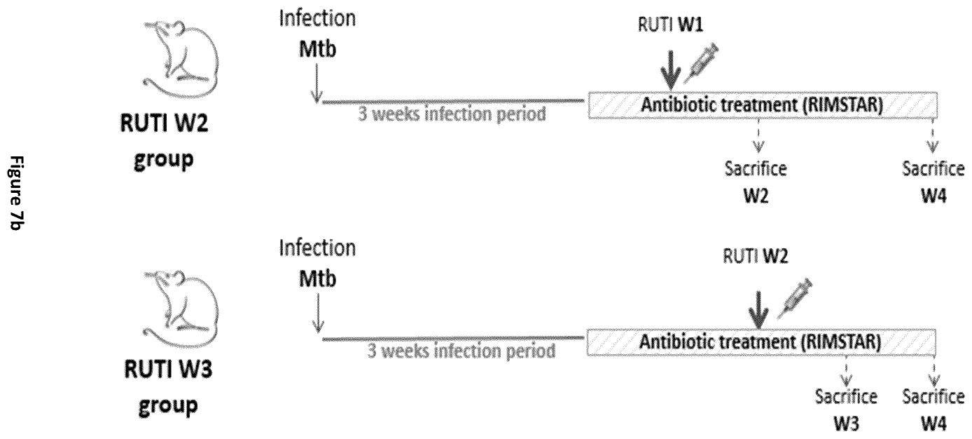

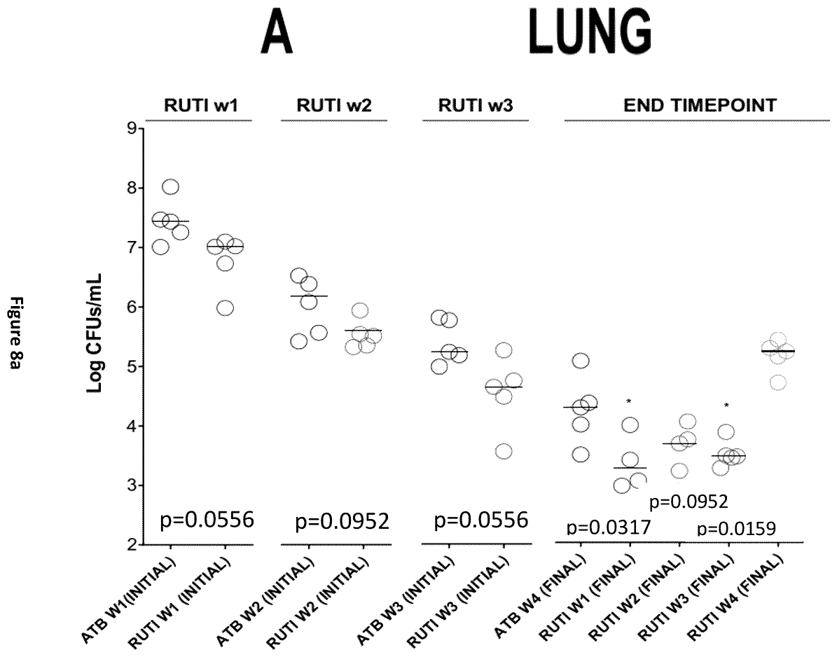

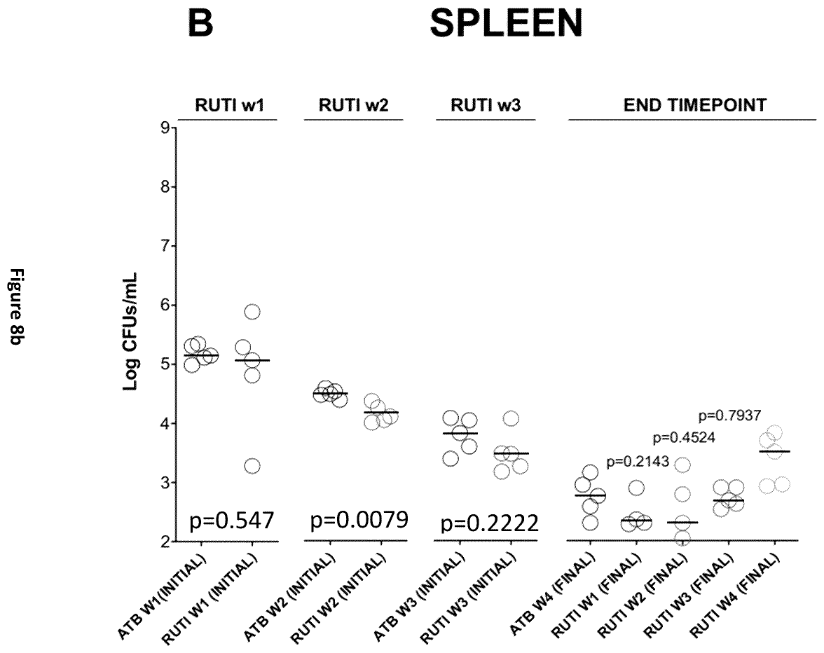

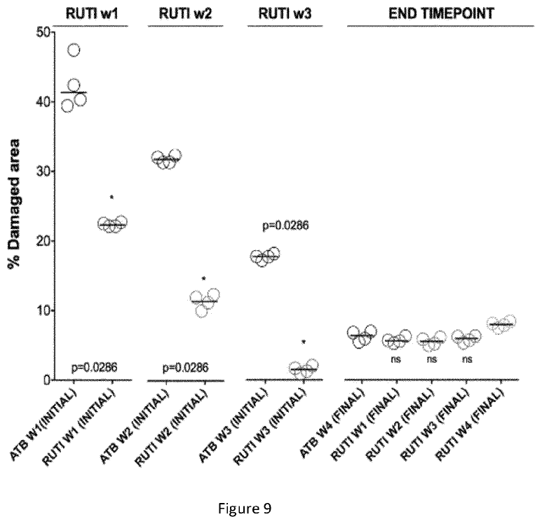

RUTI was administered subcutaneously to C3HeB/FeJ infected mice to test their effect in an active TB infection model. The bacterial load in the lungs and the lung damage after RUTI vaccine at different time points after starting the ATB were lower and better than in the control group (FIG. 8, FIG. 9).

RUTI has also shown efficacy in a Mtb-infected C3HeB/FeJ mice a humanized TB model. Thus, a single dose of RUTI reduced the bacillary load in lung and spleen when it was administered at different point (e.g. 0 day, 1 or 2 weeks) during ATB 1 (see FIG. 8).

### Pathology in Lung Tissue

Data showed a significant reduction of lung damage when RUTI was in combination of chemotherapy at dose of 200 μg FCMtb. Thus, a single dose of RUTI reduced the lung damage area when it was administered at different point upon the start of ATB (see FIG. 9). No safety concern was observed in mice at any time point of vaccination.

### Cellular Immunoresponse (ELIspot)

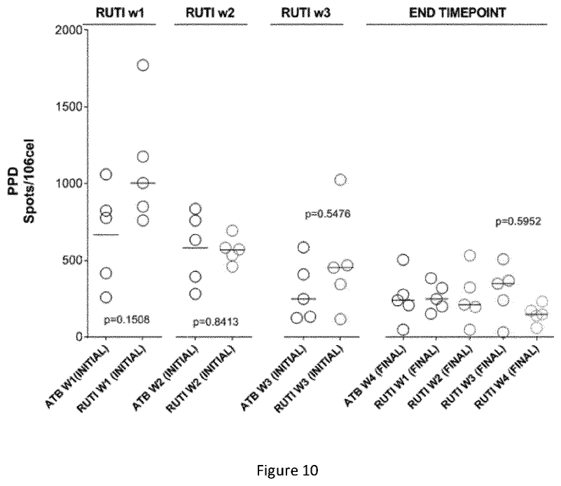

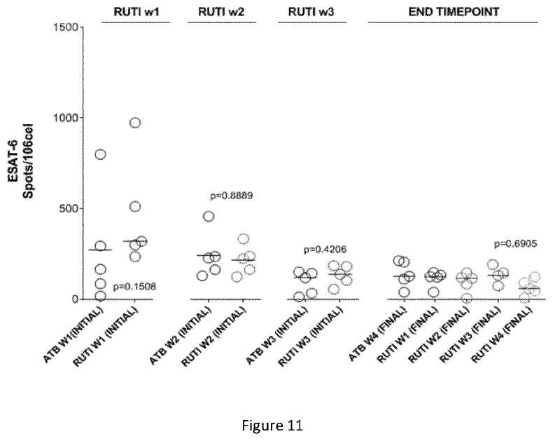

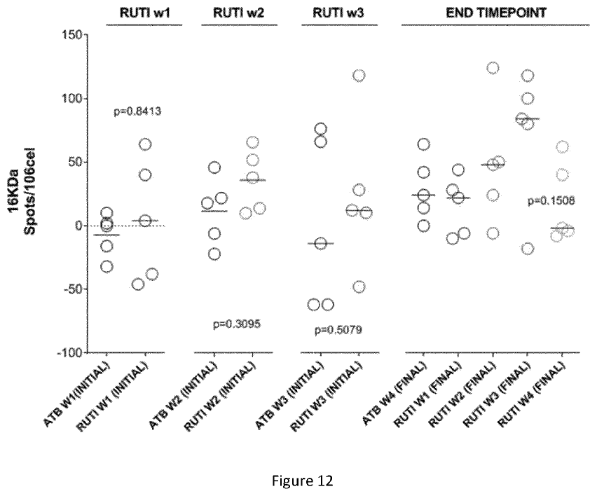

Data showed a slight induction of T cellular immunoresponse against PPD, HSP-16.3 (16 KDa) PsTS1, and ESAT-6 in RUTI vaccinated animals in comparison ATB groups (FIG. 10, FIG. 11 and FIG. 12).

### Conclusions

Using an active TB model in C3HeB/FeJ, which highly resembles TB human-liquefaction and caveated lesions, the administration of RUTI (a preferred embodiment of the liposomal formulation of the present invention) at different times after starting chemotherapy caused a reduction in the bacillary load together with a very significant reduction of the damage area in the lung. RUTI administered groups showed an induction of T-cell immune response against structural and latent antigens, the effect was mild. Thus, data shows for the first time the efficacy and safety of a therapeutic vaccine administered after the induction of active TB and while administering standard chemotherapy.

Traditionally it has been a lot of concern on therapeutic vaccination due to its potential toxicity, a concept summarized in the so called “Koch phenomenon”. The experimental TB murine model in the C3HeB/FeJ strain offers a unique opportunity to test it as it develops “human-like” lesions with liquefaction, which has a lot of parallelism with human TB. Surprisingly, RUTI administration has shown its capacity to increase the efficacy of chemotherapy both by reducing the bacillary load and the damage area in lung when administered as early as day 0 of the beginning of the chemotherapy, by showing a significant improvement in the damage area, and a reduction trend in the bacillary load that becomes significant by week 3.

Regarding the mechanism of action, RUTI is designed to induce a polyantigenic response able to survey for dormant bacilli. Even when the immune response obtained in the splenocytes using different Mtb antigens do not show significant differences with the control, it shows a tendency to increase the Th1 response against HSP16.3 antigen and PPD. In the case of ESAT-6 and 85B antigen there is an overall decrease, corresponding with the reduction of the bacillary load with time. These data aims to test immunotherapeutic vaccination in active TB patients.

In conclusion, the non-clinical results show the RUTI is efficacious (bacillary load and lung damage area) independently of when it is administered while standard chemotherapy. The results support the selected time point vaccination from 0 days upon the start of the chemotherapy. In addition, the reduction of the bacillary load is maintained over time with respect to the chemotherapy.

### Example 10: Efficacy of Liposome Formulations (Drug Product) Comprising Fragments from a Mycobacterium tuberculosis-Complex (Drug Substance, FCMtb) in Active TB C3Heb/FeJ Mice Model, 200 μg and 25 μg Dose

The liposome formulations (drug product, Example 8) comprising fragments from a Mycobacterium tuberculosis-complex (drug substance, Examples 1-7) used in this example is the most preferred embodiment: RUTI. It is labelled correspondingly in the following examples, including all associated figures. The dosage refers to the dosage of the drug substance, FCMtb.

### Objective

RUTI was administered in different regimens to assess its activity both alone and in combination with chemotherapy. If chemotherapy was administered, different time periods were investigated, such as 4 days or 25 days.

Animals Male/Female (1:1) C3HeB/FeJ (5-7 weeks old) were obtained from The Jackson laboratory (Harbor, Maine, USA).

A chip (Anibio) identifies each mouse (IGTP-name-year). Mice were supervised daily, weighed and euthanized, if required, with isoflurane (inhalation excess), following a strict protocol, in order to ensure animal welfare.

The results presented are from a single study, using 6 (3 female and 3 males) C3HeB/FeJ mice per group.

Mice were infected intravenously with 4.0E+04 of M. tuberculosis H37Rv Pasteur strain (Batch 10 from UTE stock IGTP) via caudal vein.

RIMSTAR (HRZE) chemotherapy administration was orally administered a dose of 0.2 mL (5 mg rifampicin/mL; 2.5 mg Isoniazid/ml; 13.3 mg pyraminazide; 9.2 mg ethambutol/ml).

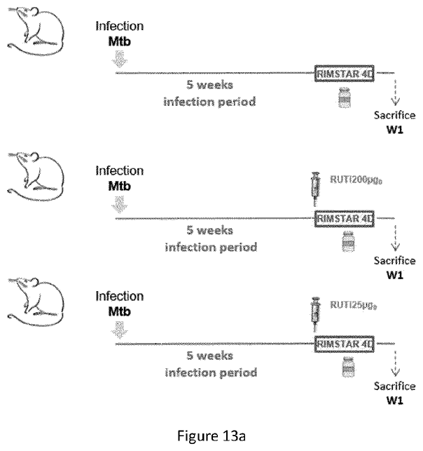

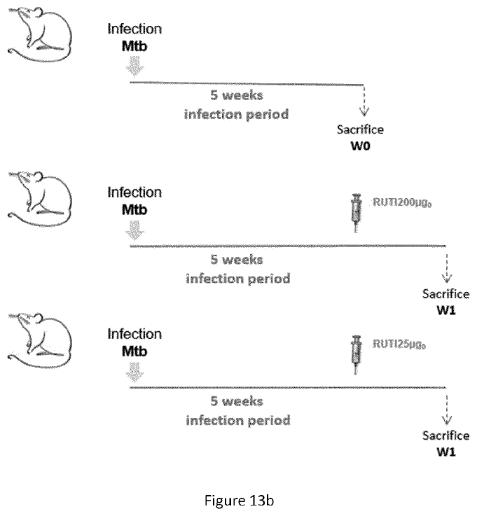

In one of the experiments, groups RUTI/sham (saline control) were administrated subcutaneously at time 0 week (Od) after starting chemotherapy and when chemotherapy was ended (see FIG. 13c). In another experiment groups RUTI/sham (saline control) were administrated subcutaneously time 0 of a shorter chemotherapy treatment (FIG. 13a). In further experiments, groups only received RUTI/sham (saline control) and no chemotherapy (FIG. 13b).

Animals were sacrificed at different time points during the experiments, for example 1 week and/or 6 weeks after RUTI administration (see FIG. 13 a/b/c). After the sacrificed the lung and the spleen was removed in order to assess the bacillary load (CFU), the lung damage and the T cell immunological response (from splenocytes). Blood was recollected and the serum was obtained and was kept in −80° C.

The effect of RUTI on the reduction of bacillary load, the improvement of lung damage and the cellular immune response (ELispot) were studied All procedures were performed in a BLS3 security facility (CMCiB) according to protocol DMAH9559, which was reviewed by the Animal Experimentation Ethics Committee of the Institut Germans Trias i Pujol (IGTP) (registered as B9900005) and approved by the Dept d′Agricultura, Ramaderia, Pesca, Alimentació i Medi Natural of the Catalan Government, according to current national and European Union legislation regarding the protection of experimental animals (Law 1997 of the Catalan Government; Spanish Royal Decree 1201/2005; and European legislation 86/609/EEC; 91/628/EEC; 92/65/EEC and 90/425/EEC).

### Detailed Experimental

Bacillary Load (CFUs/mL), lung pathology, T cell immunological response (Elispot technique) and data analysis-statistical methods, see Example 9.

### Mtb Cell Wall Fragment Containing Product:

The drug product RUTI is a vaccine presented as a dry powder for reconstitution containing 266.7 μg or 33.3 μg of FCMtb (drug substance, fragmented cells of Mtb) per vial. It is supplied in amber glass vials that comply with current Pharmacopeia European (Ph.Eur.) and United State Pharmacopeial convention (USP) requirements for glass containers for pharmaceutical use.

The Drug Substance (DS) consists in purified cell wall fragments of Mtb named FCMtb. The manufacturing process of FCMtb is obtained by cellular fragmentation of harvested bacterial cultures of Mtb (examples 1-7). Afterward the fragments cell walls are purified in presence of Triton X100 to remove the soluble components and the endotoxin-like molecules. The structure of the DS is tightly related to the characteristics of the cell wall of Mtb. Thus, DS is composed mainly by a heterogeneous spectrum of proteins and lipids from Mtb strain. RUTI, the drug product (a preferred embodiment of the liposome formulation of the present invention), is the DS liposome suspension with a charge excipient. It is presented as a dry powder for reconstitution with water for injection and it is stable at 5° C. for at least 12 months.

TABLE 7
Quantitative and qualitative composition of RUTI vaccine per vial

| | | |
|---|---|---|
| Components | Unit per vial | Function |

Drugs substance

| | | | |
|---|---|---|---|
| FCMtb | 266.7 μg | 33.3 μg | Immunogen |

Excipients

| | | | |
|---|---|---|---|
| Sucrose | 20,000.0 μg | 20,000.0 μg | Charge substance (freeze- |
| | | | drying) and cryoprotector |
| Soy lecithin ¹ | 3,383.2 μg | 422.9 μg | Liposome forming agent |
| Sodium cholate | 368.0 μg | 46.0 μg | Tensoactive |
| Sodium chloride ² | 83.2 μg | 10.4 μg | Isotonic agent |
| Ethanol ³ | q.s | q.s | Solvent |
| Water for injection ³ | q.s | q.s | Solvent |

¹ Containing Phosphatidylcholine (Not less than [NLT] 94.0%)
² Added as NaCl 0.9% solution
³ It disappears in the course of processing

For administration to mice, the investigational drug product should be reconstituted with 0.266 mL of water for injection to give a solution containing 1002.1 μg/mL of FCMtb (FCMtb 266.7 μg/vial) and 125.3 μg/ml of FCMtb (RUTI 33.3 μg/vial). The composition of the placebo to be used in the non-clinical trial presented is saline (0.9% NaCl).

A total volume of 0.200 ml of reconstituted RUTI vaccine will be administered subcutaneously in the neck area.

### Results

RUTI was administered subcutaneously to C3HeB/FeJ infected mice to test their effect in an active TB infection model. A volume of 0.2 ml (containing 4.0E+04 Mtb CFUs) were administered intravenously in tail to infect the animal. After 5 weeks, a shot of RUTI at 200 μg of FCMtb or at 25 μg of FCMtb; or Sham (saline) were administered alone and in combination with chemotherapy at time 0 (S1w). In some experiments, a second shot of RUTI at 200 μg of FCMtb or at 25 μg of FCMtb; or Sham (saline) was administered at the end of chemotherapy (28 d). In some experiments, animals were sacrificed one week after first RUTI/Sham administration or 2 weeks after second RUTI/Sham administration.

The efficacy of RUTI treatment was studied by measuring the bacillary load in lung and spleen while the safety was assessed as the percentage of lung damage by histopathology. The T immunoresponse was evaluated using ELISpot technique.

### Bacillary Load in Lung and Spleen Tissue

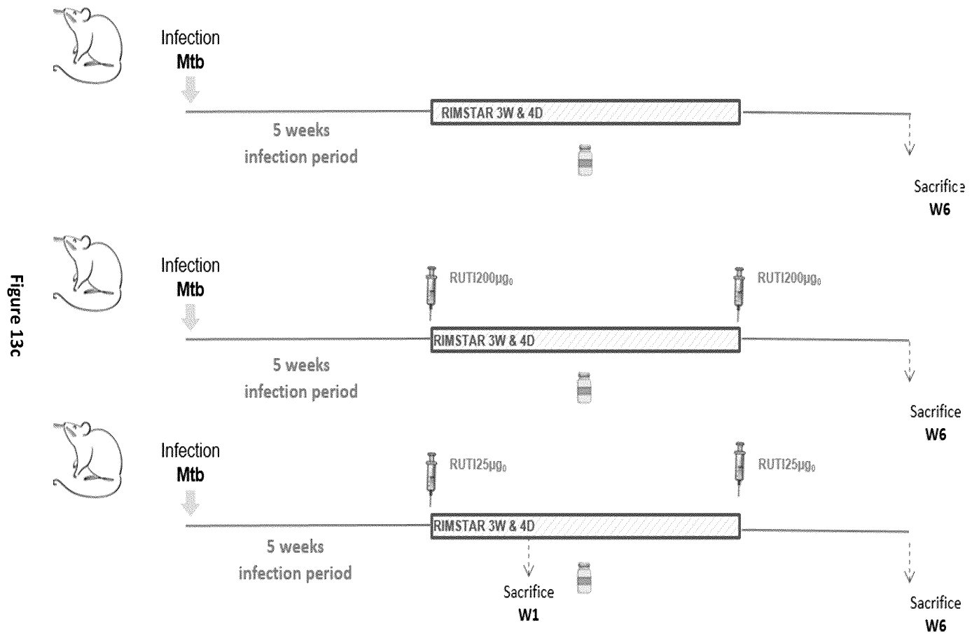

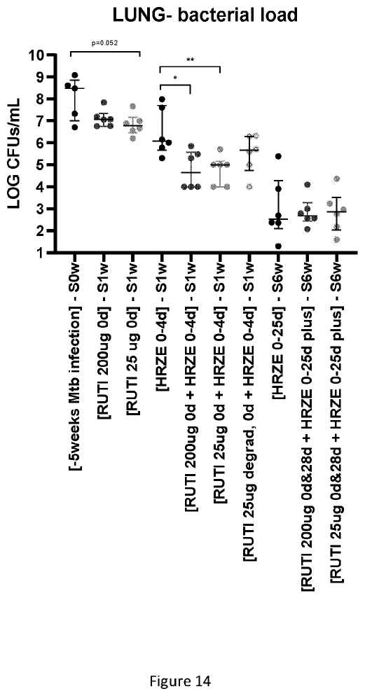

Data showed a significant reduction of bacillary load when RUTI was administered alone or in combination of chemotherapy at time 0. 200 μg of FCMtb and 25 μg of FCMtb doses of RUTI showed a similar reduction of CFUs. (see FIG. 14)

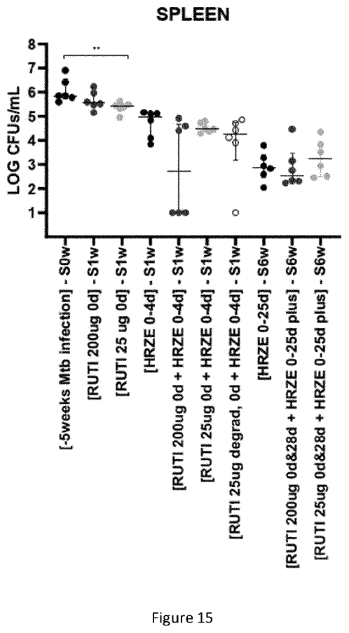

In spleen, Data showed a significant reduction of bacillary load when RUTI was administered alone at time 0 (FIG. 15).

### Pathology in Lung Tissue

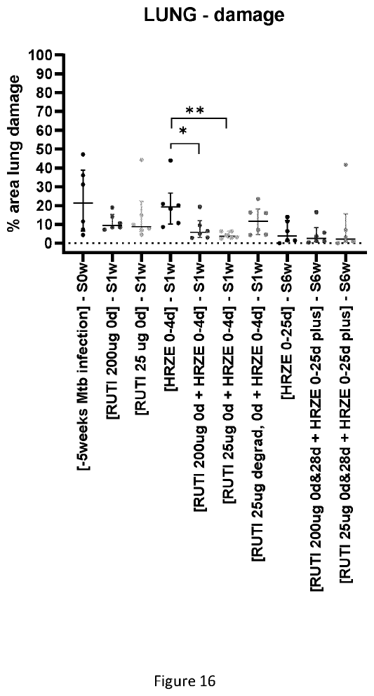

Data showed a significant reduction of lung damage when RUTI was administered alone or in combination of chemotherapy at time 0. 200 μg of FCMtb and 25 μg of FCMtb doses of RUTI showed a similar reduction of lung damage (FIG. 16).

### Cellular Immunoresponse (ELIspot)

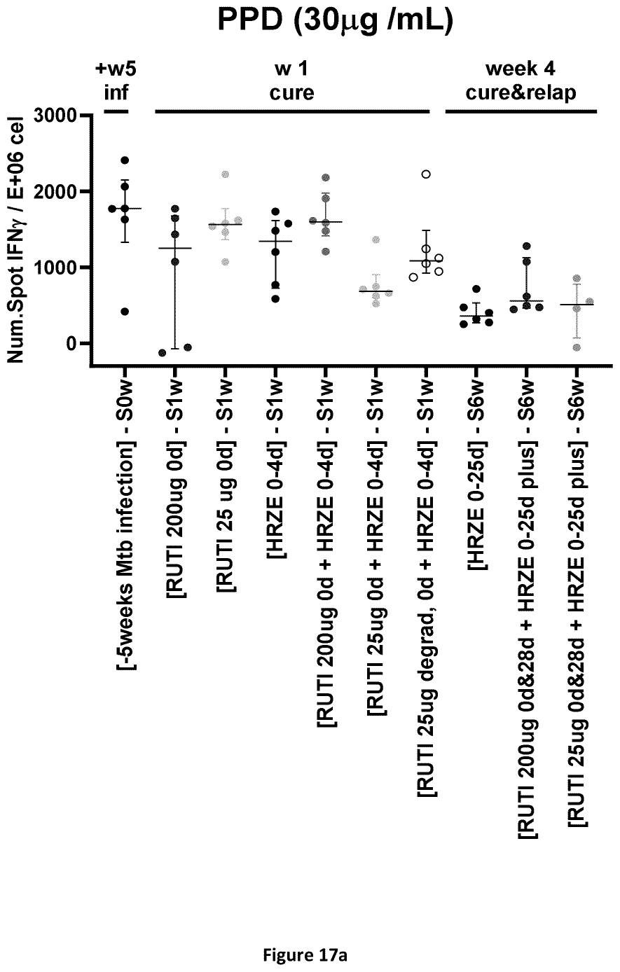

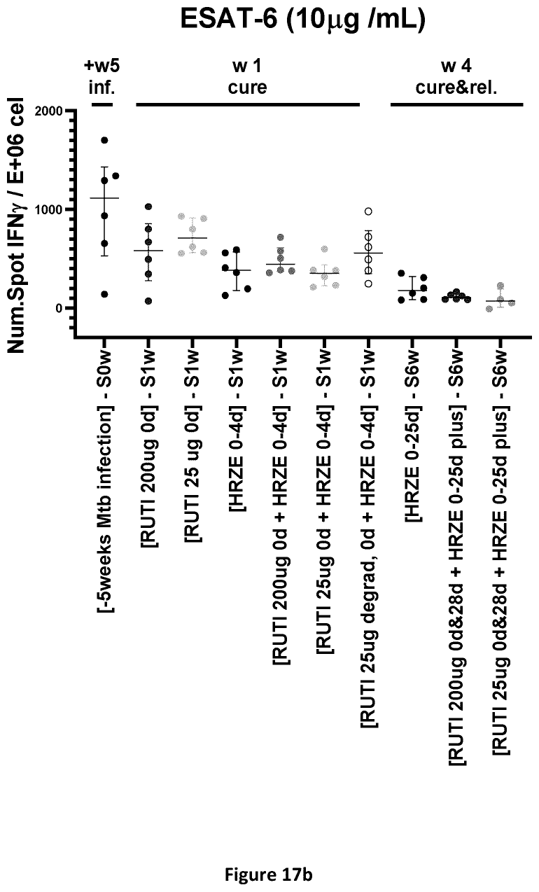

Data showed that the reduction of CFUs caused a reduction of T-cell immune response against ESAT-6 while RUTI administration did not induce specifically ESAT-6 response (see FIG. 17).

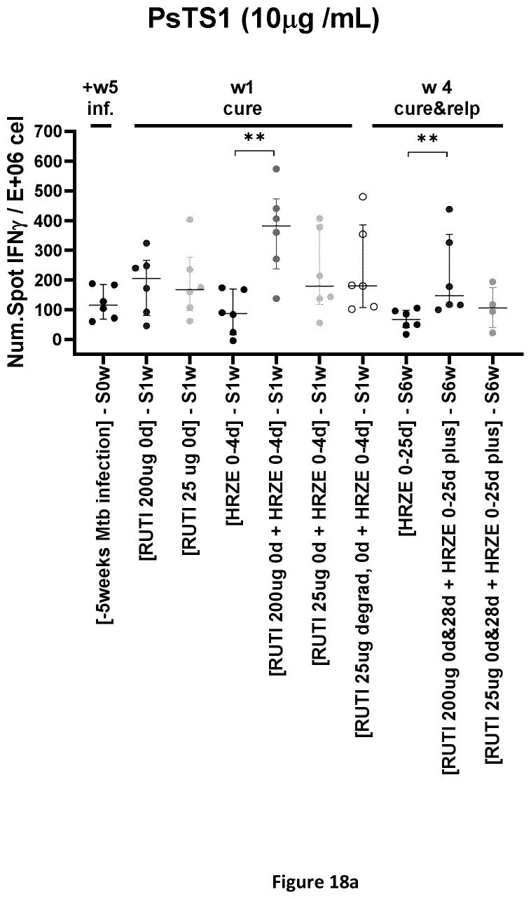

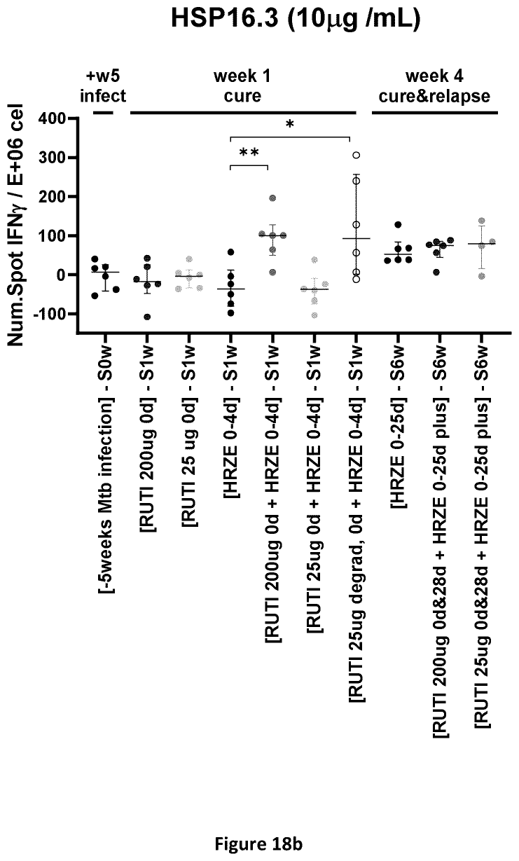

Data showed that RUTI administration induced a T-cell immunoresponse against HSP16.3 and PsTS1 T in a dose-dependent manner after a single shot. (FIG. 18).

### Conclusions

Traditionally it has been a lot of concern on therapeutic vaccination due to its potential toxicity, a concept summarized in the so called “Koch reaction”. Briefly this concept comes from the experimental experience of Robert Koch in Mtb infected guinea pigs, were the inoculation of tuberculin caused a necrotic reaction in already set infected granulomas. This reaction was the base to attribute a pathological reaction of therapeutic vaccination in patients suffering active TB ever since. The experimental TB murine model in the C3HeB/FeJ strain offers a unique opportunity to test it as it develops “human-like” lesions with liquefaction, which has a lot of parallelism with human TB.

Using an active TB model in C3HeB/FeJ, which highly resembles TB human-liquefacted lesions, the administration of RUTI alone or at time 0 of starting chemotherapy caused a reduction in the bacillary load together with a very significant reduction of the damage area in the lung. RUTI administered groups showed an induction of T-cell immune response against HSP-16.3 and PsTS1, latent antigens, but not PPD and ESAT-6.

Surprisingly, RUTI administration alone has shown its capacity to works similar to chemotherapy the first week of treatment by reducing the bacillary load and the damage area in lung. Regarding the mechanism of action, RUTI is designed to induce a polyantigenic response, but no induction of T-cell response has been observed either PPD, ESAT-6, HSP16-3 or PsTS1. In the case of ESAT-6 there is an overall decrease, corresponding with the reduction of the bacillary load with time. The lack of increased immune response when administering RUTI alone can be explained by the already increased response in the infected mice. It can be speculated that RUTI injection allows the recruitment of extra specific lymphocytes to stop the progression of endogenous reactivation, and that the technique used has not enough sensitivity to record a difference. This is in contrast with what we see with chemotherapy, as it sudden stops the immune response by killing the bacilli, stopping the positive feedback with the lymphnodes in order to stimulate the lymphocytic proliferation. In this case, the mobilization caused by RUTI can be detected and make the difference.

Additionally, data confirms the efficacy and safety of a therapeutic vaccine after the induction of active TB in combination of chemotherapy. The effect of RUTI has been observed in dose dependent manner (200 μg and 25 μg dose) after a single shot at time 0 of the standard chemotherapy. Regarding the mechanism of action, RUTI is designed to induce a polyantigenic response able to surveil for dormant bacilli. It shows a statistically significant increase of the Th1 response against HSP16.3 antigen and PsTS1. In the case of ESAT-6 there is an overall decrease, corresponding with the reduction of the bacillary load with time.

It is also important to note the lack of effect after the second administration of RUTI at the end of the chemotherapy. This is probably due to the low bacillary load. In this case the more feasible would be to wait at least for 6 weeks to look for the effect of RUTI vaccination against reactivation, as it has been already demonstrated. This “Cornell-like” methodology is what has been usually done for testing therapeutic vaccines. The data obtained thus just confirm previous data.

In conclusion, the non-clinical results show the RUTI is efficacious (bacillary load and lung damage area) independently of when it is administered alone or in combination of the standard chemotherapy at the beginning of the treatment. The results support the selected time point vaccination from 0 days upon the start of the chemotherapy. In addition, the reduction of the bacillary load is maintained over time with respect to the chemotherapy.

### Example 11: Phase IIb in Patients with MDR-TB and DS-TB

The RUTI® vaccine stimulates host immune effectors to achieve elimination of actively replicating bacilli and persistent bacilli as adjunctive therapy of antibiotic treatment in TB. The first step is assuring that the RUTI® vaccine is safe and immunogenic and reduces the time to negativity of sputum cultures in patients with DS-TB and MDR-TB.

Based on the available data, a phase IIb is ongoing in India. The study is titled “Double-Blind, Randomized, Placebo-Controlled Phase IIb Clinical Trial to Investigate the Efficacy of RUTI® Therapeutic Vaccination as adjuvant of Tuberculosis chemotherapy” and was approved by Drugs Controller General of India (DCGI) on 20 Jan. 2020.

The main objectives of the study are:
1. To evaluate the time to Sputum Culture Conversion of the RUTI® vaccine (25 μg FCMtb) in patients with DS-TB and MDR-TB favourably responding to standard TB treatment as evidenced by clinical response.
2. To explore the safety and immunogenicity of the RUTI® vaccine (25 μg FCMtb) in patients with DS-TB and MDR-TB favourably responding to standard TB treatment.

The clinical trial is designed as a prospective, randomized, double-blind, multicentre, placebo-controlled clinical phase IIb trial to evaluate efficacy of RUTI® vaccine in DS- and MDR-TB patients favourably responding to standard MDR-TB treatment. Time point of vaccination starts upon completion of 1 week of standard DS-TB treatment (cohort A), and another cohort of patients will be vaccinated upon completion of 1 month of standard MDR-TB treatment (cohort B). All the patients will be followed up to the end of the treatment.

The study plans to enrol 140 adult TB patients (age >18 years) with culture confirmed DS-TB (90 patients) and MDR-TB (50 patients), and without any disease that could compromise the assessment of the response to the vaccination, or would increase the risk of adverse events. Currently all of the 90 DS-TB patients and 20 MDR-TB patients have been recruited and randomized 1:1 to receive one subcutaneous shot of 25 μg RUTI® vaccine or placebo at 1 week or 4 weeks respectively after the beginning of standard MDR-TB treatment. No significant safety concerns have emerged, only some local reactions have observed.

Two further studies investigating the safety of a 25 μg RUTI® vaccine against a placebo administered at the same time as standard treatment is started or 16 weeks after starting antibiotic treatment are underway. No significant safety concern have emerged.

For example, the former study explores the efficacy and safety of the concomitant administration of RUTI® immunotherapy with the standard treatment in patients with TB (CONSTAN).

The main objectives of the study are:
1. To evaluate the Early Bactericidal activity (from 0 to 14 days) of the RUTI® vaccine (25 μg FCMtb) in patients with DS-TB when RUTI is given at the beginning of ATB treatment (Day 0).
2. To evaluate the safety and tolerability of the RUTI® vaccine (25 μg FCMtb) in patients with DS-TB when RUTI is given at the beginning of ATB treatment (Day 0).

### REFERENCES

Andersen P., 1997, Scand J. Immunol.; 45(2):115-31
Brennan, Tuberculosis (Edinburgh), 2003, 83(1-3), 91-97
Chouldhary et al., 2018, Journal Immunology, 200:3053-3066
Geisel et al., 2005, J. Immunol.; 174(8):5007-15
Meier et al., 2018, Frontiers in Immunology Vol9 Article 2476
Renshaw, et al., 2005, EMBO Journal 24, 2491-2498
Rodriguez-Hernandez, et al. 2020, Biomed and Biotechnol 21(11):856-870
Singh, et al., 2005, Clin. Diagn. Lab. Immunol. 12(2), 354-358
Stewart et al. 2005, Infect. Immun., 73(10):6831-7
Wang et al., 2007, J. Mol. Biol., 366(2):375-81

1. A method of treating active tuberculosis in a subject in need thereof, comprising administering to the subject a liposome formulation comprising:
(a) fragments from a Mycobacterium tuberculosis-complex (MTB-C) strain,
(b) a liposome forming agent,
(c) 1 to 20% (w/v) sucrose,
wherein the z-average size of the particles is 150 nm or less, as determined by dynamic light scattering and the polydispersity index of the particles is 0.400 or less.

2. The method according to claim 1, wherein the Mycobacterium tuberculosis-complex (MTB-C) strain is a virulent Mycobacterium tuberculosis-complex (MTB-C) strain, preferably the MTB-C strain NCTC 13536, deposited in 2010 at the NCTC in London.

3. The method according to claim 1, wherein the formulation additionally comprises:
(a) a tensioactive agent, and/or
(b) one or more non-ionic surfactants.

4. The method according to claim 1, wherein the liposome forming agent is a hydrogenated, partially hydrogenated or non-hydrogenated phospholipid.

5. The method according to claim 1, wherein the formulation comprises at least two of the following:
(a) a first polypeptide having a molecular weight of about 70 kDa as measured following electrophoresis on a sodium dodecylsulfate (SDS) polyacrylamide gel, wherein the first polypeptide has a mass fingerprint similar to a mass fingerprint of M. tuberculosis HSP70 protein (Rv0350),
(b) a second polypeptide having a molecular weight of about 38 kDa as measured following electrophoresis on a sodium dodecylsulfate (SDS) polyacrylamide gel, wherein the second polypeptide has a mass fingerprint similar to a mass fingerprint of M. tuberculosis 38 kDa protein (Rv 0934),
(c) a third polypeptide having a molecular weight of about 30-34 kDa as measured following electrophoresis on a sodium dodecylsulfate (SDS) polyacrylamide gel, wherein the third polypeptide has a mass fingerprint similar to a mass fingerprint of M. tuberculosis Ag85 complex protein (Rv 1866c-Rv 3804c),
d) a fourth polypeptide having a molecular weight of about 16 kDa as measured following electrophoresis on a sodium dodecylsulfate (SDS) polyacrylamide gel, wherein the fourth polypeptide has a mass fingerprint similar to a mass fingerprint of M. tuberculosis HSP 16.3 protein (Rv2031c)
(d) a fifth polypeptide having a molecular weight of about 10 kDa as measured following electrophoresis on a sodium dodecylsulfate (SDS) polyacrylamide gel, wherein the fifth polypeptide has a mass fingerprint similar to a mass fingerprint of M. tuberculosis CFP10 protein (Rv3874), and
(e) a sixth polypeptide having a molecular weight of about 6 kDa as measured following electrophoresis on a sodium dodecylsulfate (SDS) polyacrylamide gel, wherein the sixth polypeptide has a mass fingerprint similar to a mass fingerprint of M. tuberculosis ESAT-6 protein (Rv3875).

6. The method of claim 1, wherein the liposome formulation of comprises a pharmaceutically acceptable carrier or diluent, and/or a pharmaceutically acceptable adjuvant.

7. The method of claim 1, wherein the active tuberculosis is drug susceptible tuberculosis, Rifampicin resistant tuberculosis, multidrug-resistant tuberculosis or extensive drug resistant tuberculosis.

8. The liposome method of claim 1, wherein the liposome formulation is administered once or twice.

9. The method of claim 1, wherein the liposome formulation is administered in the absence of chemotherapy.

10. The method of claim 1, wherein the liposome formulation is administered before or concomitant with chemotherapy.

11. The method of claim 10, wherein the chemotherapy lasts for 4 weeks.

12. The method of claim 10, wherein the first dose of the liposome formulation or pharmaceutical composition is administered within four weeks of the first dose of chemotherapy.

13. The method of claim 10, wherein the chemotherapy is antibiotic treatment.

14. The method of claim 13, wherein the antibiotic treatment comprises at least one of ethambutol, isoniazid, pyrazinamide, rifampicin, streptomycin, amikacin, kanamycin, capreomycin, viomycin, enviomycin, ciprofloxacin, levofloxacin, moxifloxacin, ethionamide, prothionamide or cycloserineterizidone.

15. The method of claim 1, wherein the liposome formulation or a pharmaceutical composition comprising the liposome formulation is administered at a dose of 5-200 μg of FCMtb per dose.

16. The method according to claim 4, wherein the liposome forming agent is soy lecithin.

17. The method of claim 12, wherein the first dose of the liposome formulation or pharmaceutical composition is administered together with the first dose of chemotherapy.

18. The method of claim 14, wherein the antibiotic treatment comprises RIMSTAR.
